(19) United States
(12) Patent Application Publication
(10) Pub. No.: US 2025/0115540 A1
(43) Pub. Date: Apr. 10, 2025

(54) SYSTEMS AND METHODS TO PURIFY CRUDE ETHANOL FOR HIGH PURITY ETHANOL

(71) Applicant: LITZEN PROCESS CONSULTING, INC., RAPID CITY, SD (US)

(72) Inventors: David Litzen, Rapid City, SD (US); Sam Papendick, Rapid City, SD (US)

(21) Appl. No.: 18/908,153

(22) Filed: Oct. 7, 2024

Related U.S. Application Data
(60) Provisional application No. 63/588,157, filed on Oct. 5, 2023.

(51) Int. Cl. C07C 29/80

(52) U.S. Cl. CPC C07C 29/80

(57) ABSTRACT

Chemical purification apparatus(es), system(s), and/or method(s) include the use of two towers and/or columns. The apparatus(es), system(s), and/or method(s) can be vacuum-operated and can be used to purify ethanol. The apparatus(es), system(s), and/or method(s) are capable of receiving crude ethanol and refining said crude ethanol until it is essentially pure and suitable for use in the pharmaceutical, beverage, and laboratory industries. Such crude ethanol can originate from petrochemical and/or agricultural sources. The apparatus(es), system(s), and/or method(s) are capable of producing practically pure ethanol at a high quality with low energy consumption per unit volume.

### CROSS REFERENCE TO RELATED APPLICATIONS

This application claims priority under 35 U.S.C. § 119 to provisional patent application U.S. Ser. No. 63/588,157, filed Oct. 5, 2023 The provisional patent application is herein incorporated by reference in its entirety, including without limitation, the specification, claims, and abstract, as well as any figures, tables, appendices, or drawings thereof.

### TECHNICAL FIELD

The present disclosure relates generally to apparatus(es), method(s), and/or system(s) having applications in at least the chemical purification industry. More particularly, but not exclusively, the present disclosure relates to apparatus(es), method(s), and/or system(s) for purification of crude ethanol into high purity ethanol.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

The industrial ethanol market in the United States, as well as worldwide, is massive. It has been reported that the market size of industrial ethanol in the United States alone is at least $34.46 billion with 15.5-17 billion gallons of annual production capacity. It has been further reported in recent years that the United States has about 465 million gallons of capacity of ethanol either under expansion or construction. The use of industrial ethanol is broad. Industries using industrial ethanol are fuel, food and beverage, cosmetics, personal care, electronics, pharmaceuticals, printing inks, chemical intermediates for polymers, paints and coatings, as well as others. Additionally, with recent interest in converting ethanol into sustainable aviation fuel (SAF), the market outlook is strong.

Fermentation processes differ depending on whether the ethanol is used for fuel or for other industries, such as food and beverage. The fermentation process generally uses brewer's yeast to accomplish the conversion of sugar (glucose derived from corn starch) to ethanol and carbon dioxide. In a dry mill corn ethanol plant (used for producing fuel ethanol) the primary objective is to maximize sugar conversion to ethanol and carbon dioxide, whereas beverage alcohol producers also target certain flavor and odor bodies in the final alcohol product. As a result of the differing approaches to fermentation, the formation of either more or less fermentation byproducts (i.e., impurities that can also be referred to as congeners) or perhaps even different processing byproducts are likely present in fuel alcohol. The additional fermentation byproducts are due most likely to stresses on the yeast (a living organism) as their source of food (sugar) is diminished near the end of the fermentation cycle in a fuel ethanol plant.

Where approximately 95% industrial ethanol is used for fuel, it is also used in the pharmaceutical industry for chemical processing, in the beverage industry as a neutral spirit, and in other laboratory industries. Industrial ethanol produced from either petrochemical or agricultural feedstocks inevitably contains trace impurities that can be above standard levels for specialized end use (such as pharmaceutical or beverage use). Due to the near boiling point of these impurities, traditional distillation techniques have struggled to purify the ethanol for end use in the pharmaceutical, beverage, and laboratory industries without the expenditure of significant energy and/or significant investment in energy recovery.

Most existing purification processes in the beverage alcohol industry start by stripping ethanol from the aqueous fermentation broth and rectifying the stripped ethanol to remove water and concentrate ethanol. However, because of the ethanol/water azeotrope, the ethanol can only be concentrated using conventional distillation to approximately 95 wt % ethanol, with the remaining 5% being water (i.e., 190 proof). Water also forms azeotropes with many processing impurities, making it difficult in some cases to remove those impurities via conventional distillation. Differentiating from the beverage alcohol industry, fuel alcohol must be dehydrated to near 200 proof (i.e., ethanol with no water present) to enable blending with gasoline.

In particular to ethanol produced from starch or lignocellulose, the fermentation and processing impurities (i.e. n-propanol, acetal, acetaldehyde, methanol, etc.) have proven difficult to completely remove from ethanol. Traditional processes run a “beer” or fermented broth through sequential distillations, batchwise, until purity specifications are met. Other processes take crude ethanol from petroleum feedstocks and produce a finished ethanol composition where acetal is reduced to less than 50 parts per million (ppm) and n-propanol less than 500 ppm (see, e.g., U.S. Pat. No. 8,318,988). These levels may still be too high for USP or beverage markets, and certainly for reagent grade, where impurity levels need to be <10 ppm. The impurities in agricultural ethanol are generally different from petrochemical ethanol due to fermentation byproducts. This agricultural ethanol can show impurities with the sum of acetaldehyde and acetal over 2000 ppm and n-propanol ranging 80-700 ppm. Butanol impurities can also show up in agricultural ethanol over 1000 ppm.

In general, extractive distillation techniques have been used to purify crude ethanol to have impurities below 1 ppm (see, e.g., U.S. Pat. No. 3,445,345). This process is both energy and capital intensive. There are no readily known available patents, prior art, or information available in the public domain as it relates to specifically converting dry fuel ethanol to beverage alcohol.

Current ethanol distillation facilities and processes consume large amounts of energy in order to function. For example, current ethanol purification and/or distillation techniques consume about 46 lbs steam/gal 192 proof spirit (i.e., 39,000 BTU/gallon). Also, current ethanol purification and/or distillation techniques often have a coefficient of performance (CoP) less than 8.

Thus, there exists a need in the art for an apparatus, system, and/or method which can purify chemicals, such as ethanol, in a cost-effective manner wherein the purified ethanol is suitable for use in the pharmaceutical, beverage, and laboratory industries. There exists a further need in the art for an apparatus, system, and/or method which can purify chemicals, such as ethanol, without the use of filtration and/or or catalysts. There exists a further need in the art for an apparatus, system, and/or method which can purify chemicals, such as ethanol, while limiting energy consumed by the purification apparatus, system, and/or method such that the purification is energy-efficient and has a relatively high CoP. There exists a further need in the art for an apparatus, system, and/or method which can purify chemicals, such as ethanol, such that the purified chemical is practically absolutely pure and/or such that impurity levels are less than 10 ppm. There exists a further need in the art for an apparatus, system, and/or method for purifying fuel ethanol such that the purified ethanol achieves a quality suitable for use as beverage alcohol. There exists a further need in the art for an apparatus, system, and/or method for purifying ethanol in an environmentally friendly manner.

### BRIEF SUMMARY

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art.

It is a further object, feature, and/or advantage of the present disclosure to provide an apparatus, system, and/or method capable of producing the purest ethanol of the highest quality with the lowest energy consumption per unit volume.

It is a further object, feature, and/or advantage of the present disclosure to provide an apparatus, system, and/or method capable of purifying ethanol such that the purified ethanol is suitable for use in the pharmaceutical, beverage, and laboratory industries.

It is a further object, feature, and/or advantage of the present disclosure to provide an apparatus, system, and/or method capable of purifying chemicals, such as ethanol, in a cost-effective manner.

It is a further object, feature, and/or advantage of the present disclosure to provide an apparatus, system, and/or method capable of purifying chemicals, such as ethanol, with or without the use of filtration and/or catalysts.

It is a further object, feature, and/or advantage of the present disclosure to provide an apparatus, system, and/or method which can purify chemicals, such as ethanol, while limiting energy consumed by the purification apparatus, system, and/or method such that the purification is energy-efficient and has a relatively high CoP.

It is a further object, feature, and/or advantage of the present disclosure to provide an apparatus, system, and/or method capable of purifying chemicals, such as ethanol, such that the purified chemical is practically absolutely pure and/or such that impurity levels of the purified chemical are less than 10 ppm.

It is a further object, feature, and/or advantage of the present disclosure to provide an apparatus, system, and/or method which can purify fuel ethanol such that the purified ethanol achieves a quality suitable for use as beverage alcohol.

It is a further object, feature, and/or advantage of the present disclosure to provide an apparatus, system, and/or method which can purify chemicals, such as ethanol, in an environmentally friendly manner.

It is still yet a further object, feature, and/or advantage of the present disclosure to provide an apparatus, system, and/or method utilizing a multi-section, vacuum-operated approach to purify chemicals, such as ethanol.

The apparatus(es), system(s), and/or method(s) disclosed herein can be used in a wide variety of applications. For example, since the use of industrial ethanol is prevalent in so many industries, the disclosed apparatus(es), system(s), and/or method(s) are applicable, useful, and provide advantages in at least those industries. Such industries include, but are not limited to, fuel, food and beverage, cosmetics, personal care, electronics, pharmaceuticals, printing inks, chemical intermediates for polymers, and paints and coatings. Further, while the present disclosure focuses mainly on the disclosed apparatus(es), system(s), and/or method(s) being used with ethanol, the apparatus(es), system(s), and/or method(s) disclosed herein could be used for purification of any suitable chemical.

It is preferred the apparatus(es), system(s), and/or method(s) be safe, cost effective, and durable. For example, the apparatus(es), system(s), and/or method(s) described herein eliminate aspects traditionally used in chemical purification including, but not limited to, the use of extractant and/or catalysts, which increases cost-effectiveness of the apparatus(es), system(s), and/or method(s) described herein. Additionally, the apparatus(es), system(s), and/or method(s) described herein limit energy consumed during the purification/distillation process which leads to the apparatus(es), system(s), and/or method(s) described herein as being more cost-effective. Additionally, the apparatus(es) and/or system(s) described herein can be adapted to resist thermal transfer, electric conductivity, and/or failure (e.g. cracking, crumbling, shearing, creeping) due to excessive and/or prolonged exposure to heat and/or cold as well as tensile, compressive, and/or balanced forces acting on the apparatus(es) and/or system(s).

It is preferred the apparatus(es), system(s), and/or method(s) described herein be environmentally friendly. For example, crude ethanol that enters the disclosed system and is not converted to purified ethanol can be purged from the system and can still be used in the fuel ethanol industry. This eliminates and/or limits loss of ethanol product. Additionally, the disclosed apparatus(es), system(s), and/or method(s) can limit energy consumption and can be entirely operated via electricity or other non-carbon energy sources such as geothermal energy. Thus, the disclosed apparatus(es), system(s), and/or method(s) can be entirely run on green power.

Methods can be practiced which facilitate use, manufacture, assembly, maintenance, and/or repair of any apparatus, system, and/or method which accomplishes some or all of the previously stated objectives.

The apparatus(es) and/or method(s) described herein can be incorporated into systems which accomplish some or all of the previously stated objectives. Additionally, the system(s) described herein can be incorporated into larger designs which accomplish some or all of the previously stated objectives.

According to some aspects of the present disclosure, a system for chemical purification comprises three distillation sections: a first section is configured to receive a crude chemical and produce a heavy and light cut; a second section operationally connected to the first section, wherein the second section is configured to receive the light cut and produce a light purge and product fraction; a third section operationally connected to the first section, wherein the third section is configured to receive the heavy cut and produce a heavy purge and product fraction; wherein the product fractions combine into a purified chemical; wherein each of the first, second, and third sections operate based on independently adjustable tower operations; and wherein the system is controlled by an algorithm wherein the algorithm comprises: determining a quality of the crude chemical; determining a target quality for the purified chemical; and adjusting the tower operations so that the purified chemical achieves the target quality.

According to some additional aspects of the present disclosure, the first section is a prefractionation section configured to receive the crude chemical at or near a middle portion and further configured to split heavy and light impurities wherein fluid comprising the heavy impurities and fluid comprising the light impurities are output from opposite ends of the first section.

According to some additional aspects of the present disclosure, the second and third sections are considered a finishing tower configured to receive the fluid comprising light impurities from the first section outlet as a vapor, saving energy, and the fluid comprising the heavy impurities from the first tower at a second outlet as a liquid.

According to some additional aspects of the present disclosure, the finishing tower is configured to output additional fluid comprising light impurities from the first end and output additional fluid comprising heavy impurities from the second end.

According to some additional aspects of the present disclosure, the finishing tower is configured to produce the product, or a heart cut, to beget the purified chemical, and wherein the finishing tower is further configured to output the purified chemical from a center portion of the second tower.

According to some other aspects of the present disclosure, a system for chemical purification comprises: a first tower, wherein the first tower is configured to receive a crude chemical; a second tower operationally connected to the first tower, wherein the second tower is configured to output a purified chemical; wherein each of the first and second towers operate based on independently adjustable tower operations; and wherein the system is controlled by an algorithm wherein the algorithm comprises: determining a quality of the crude chemical; determining a target quality for the purified chemical; and adjusting the tower operations so that the purified chemical achieves the target quality.

According to some additional aspects of the present disclosure, the first tower is a prefractionation tower configured to receive the crude chemical at or near a middle portion and further configured to split heavy and light impurities wherein fluid comprising the heavy impurities and fluid comprising the light impurities are output from opposite ends of the first tower.

According to some additional aspects of the present disclosure, the system is configured to identify categories and/or species of the heavy and light impurities, and wherein the system is further configured to set and/or identify boundaries in terms of percentages and/or concentrations of the heavy and light impurities.

According to some additional aspects of the present disclosure, the second tower is a finishing tower configured to receive the fluid comprising light impurities from the first tower at a first end and the fluid comprising the heavy impurities from the first tower at a second end.

According to some additional aspects of the present disclosure, the second tower is configured to output additional fluid comprising light impurities from the first end and output additional fluid comprising heavy impurities from the second end.

According to some additional aspects of the present disclosure, the second tower is configured to perform a heart cut to beget the purified chemical, and wherein the second tower is further configured to output the purified chemical from a center portion of the second tower.

According to some additional aspects of the present disclosure, the adjustable tower operations comprise depth of vacuum, distillate-to-feed ratio, reflux-to-distillate ratio, water addition to the crude chemical fed into the first tower, and bottoms rate.

According to some additional aspects of the present disclosure, the algorithm comprises a feedforward algorithm, a feedback algorithm, and/or incorporates feedforward and feedback characteristics.

According to some additional aspects of the present disclosure, gas chromatography is utilized to measure and/or determine the quality of the crude chemical and an actual quality of the purified chemical.

According to some additional aspects of the present disclosure, the system utilizes mechanical vapor recompression.

According to some additional aspects of the present disclosure, each of the first and second towers are packed and/or trayed.

According to some additional aspects of the present disclosure, the crude chemical is crude ethanol.

According to some additional aspects of the present disclosure, the purified chemical is purified ethanol wherein the purified ethanol is nearly absolutely pure.

According to some additional aspects of the present disclosure, the purified chemical comprises impurity levels that are less than 10 parts per million.

According to some other aspects of the present disclosure, a method of refining a chemical comprises: determining a quality of a crude chemical; determining a target quality for a refined chemical; adjusting column operations of a first and second column so that the refined chemical achieves the target quality; inputting the crude chemical into the first column; performing prefractionation of the crude chemical; separately outputting fractioned portions of the crude chemical from the first column; separately inputting the fractioned portions of the crude chemical into the second column; refining the fractioned portions of the crude chemical via the second column to beget a refined chemical; and outputting the refined chemical from the second column.

According to some additional aspects of the present disclosure, performing prefractionation of the crude chemical comprises splitting the crude chemical into fluid comprising heavy impurities and fluid comprising light impurities.

According to some additional aspects of the present disclosure, refining the fractioned portions of the crude chemical comprises performing a heart cut of the fluid comprising the heavy impurities and the fluid comprising the light impurities.

According to some additional aspects of the present disclosure, the method further comprises customizing the target quality for the refined chemical.

According to some additional aspects of the present disclosure, the method further comprises verifying an actual quality of the refined chemical to ensure compliance with the target quality.

According to other some aspects of the present disclosure, a chemical distillation assembly comprises: a first tower, wherein the first tower is configured to receive a crude chemical and perform prefractionation of said crude chemical; a second tower operationally connected to the first tower, wherein the second tower is configured to receive fractioned crude chemical from the first tower, distill the fractioned crude chemical to obtain a distilled chemical, and to output the distilled chemical; wherein each of the first and second towers operate based on independently adjustable tower operations; and wherein the assembly is controlled by an algorithm wherein the algorithm comprises: measuring a quality of the crude chemical; acquiring a target quality for the distilled chemical wherein the target quality for the distilled chemical is based on user input; and automatically adjusting the tower operations so that the distilled chemical achieves the target quality.

These and/or other objects, features, advantages, aspects, and/or embodiments will become apparent to those skilled in the art after reviewing the following brief and detailed descriptions of the drawings. Furthermore, the present disclosure encompasses aspects and/or embodiments not expressly disclosed but which can be understood from a reading of the present disclosure, including at least: (a) combinations of disclosed aspects and/or embodiments and/or (b) reasonable modifications not shown or described.

### BRIEF DESCRIPTION OF THE FIGURES

Several embodiments in which the present disclosure can be practiced are illustrated and described in detail, wherein like reference characters represent like components throughout the several views. The figures are presented for exemplary purposes and may not be to scale unless otherwise indicated. Further, the data presented in the figures is also for purpose of illustrating an example and is not limiting to the inventions disclosed herein.

FIG. 1 shows a schematic of a chemical purification, distillation, and/or refinement system and/or assembly according to aspects of the present disclosure.

FIG. 2 shows another schematic of a chemical purification, distillation and/or refinement system and/or assembly according to aspects of the present disclosure.

FIG. 3 shows two perspective views of at least some components of the system of FIG. 1.

FIG. 4 shows a flow diagram of an algorithm used to control/operate at least some aspects of the system of FIG. 1 and/or the system of FIG. 2 according to aspects of the present disclosure.

FIG. 5 shows a graphical representation of composite curves of enthalpy versus temperature.

FIG. 6 shows a block diagram of a cyberinfrastructure associated with the system and/or assembly of FIG. 1 and/or the system and/or assembly of FIG. 2 according to some aspects of the present disclosure.

FIG. 7 shows a table illustrating material flows throughout a pilot distillation unit according to some aspects of the present disclosure.

FIGS. 8-12 show measured and predicted results of aspects of ethanol distillation according to some aspects of the present disclosure.

An artisan of ordinary skill in the art need not view, within isolated figure(s), the near infinite number of distinct permutations of features described in the following detailed description to facilitate an understanding of the present disclosure.

### DETAILED DESCRIPTION

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

It is further to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4%. This applies regardless of the breadth of the range.

The term “or” is synonymous with “and/of” and means any one member or combination of members of a particular list.

The term “and/of” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “about,” as used herein, refers to variation in the numerical quantity that can occur, for example, through typical measuring techniques and equipment, with respect to any quantifiable variable, including, but not limited to, concentration, mass, volume, time, length, temperature, pH, and purity. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

The term “azeotrope” as used herein refers to a mixture of liquids that has a constant boiling point because the vapor has the same composition as the liquid mixture. The boiling point of an azeotropic mixture may be higher or lower than that of any of its components. In the case of a fuel ethanol product, azeotropes are generally ‘low boiling’, meaning the boiling point of the binary mixture is lower than the boiling point of either of its two constituents.

As used herein, the “heart cut” refers to the purified ethanol which includes fluids that have a middle range of volatility and/or boiling points; for example, the heart cut can include fluid that has volatility and/or boiling points between those of the fluid comprising the light impurities and the fluid comprising the heavy impurities.

The terms “invention” or “present invention” are not intended to refer to any single embodiment of the particular invention but encompass all possible embodiments as described in the specification and the claims.

The term “substantially” refers to a great or significant extent. “Substantially” can thus refer to a plurality, majority, and/or a supermajority of said quantifiable variable, given proper context.

The term “generally” encompasses both “about” and “substantially.”

The term “configured” describes structure capable of performing a task or adopting a particular configuration. The term “configured” can be used interchangeably with other similar phrases, such as “constructed”, “arranged”, “adapted”, “manufactured”, and the like.

Terms characterizing sequential order, a position, and/or an orientation are not limiting and are only referenced according to the views presented.

The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, sub-combinations, or the like that would be obvious to those skilled in the art.

As used herein, the term “exemplary” refers to an example, an instance, or an illustration, and does not indicate a most preferred embodiment unless otherwise stated.

The terms “distillation”, “purification”, “refinement”, and any variations thereof, can be used interchangeably herein.

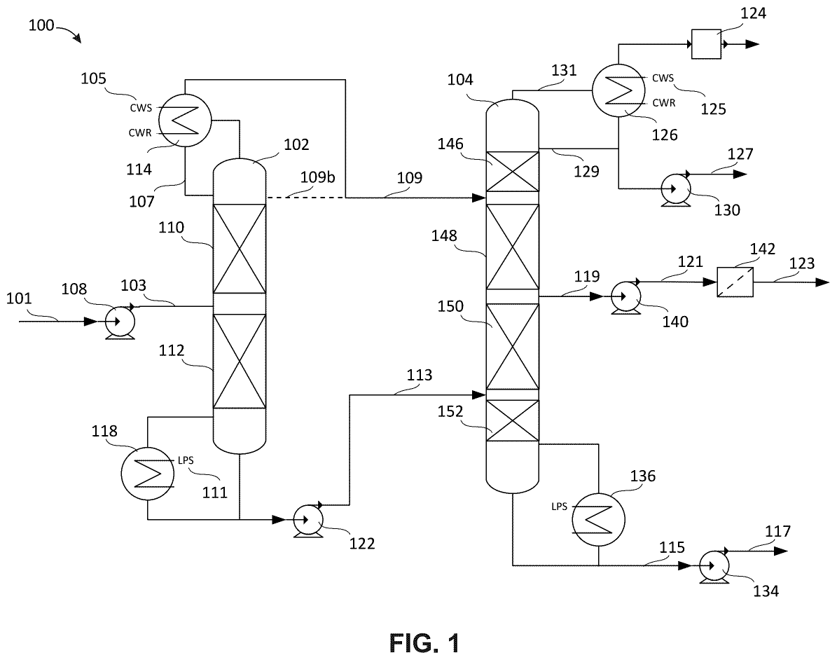

Referring now to the figures, FIG. 1 shows a schematic of a chemical purification system and/or assembly 100 according to some embodiments. While the schematic shown in FIG. 1 is generally referred to as the system 100 herein, it could also be referred to as an assembly 100. Further, while the schematic shown in FIG. 1 is referred to as a purification system 100 herein, it could be referred to as a distillation system and/or a refinement system. Additionally, while much of the present disclosure focuses on the purification of ethanol, the system 100 could be used to purify, distill, and/or refine any suitable chemical. Whenever the present disclosure refers to “ethanol”, any suitable chemical could be substituted, such as, but not limited to, methanol or propanol. It should be noted that the arrows appearing on any lines, piping, hoses, ducts, streams, channels, and/or any other passages in FIG. 1 represent the flow of fluid through said lines, piping, hoses, ducts, streams, channels, and/or passages.

As shown in FIG. 1, the purification system 100 can include two towers (which can also be referred to as columns herein), a first tower (column) 102 and a second tower (column) 104. The system 100 and/or each of the first and second towers 102, 104 can be vacuum-operated. Each of the first and second towers 102, 104 can include one or more fluid entrances and one or more fluid exits. Fluid entrances and/or exits can be a nozzle, inlet, feed, drain, pipe, and/or any other kind of suitable opening. Additionally, each of the first and second towers 102, 104 can be packed and/or trayed. For example, according to different embodiments, both towers 102, 104 could be packed, both could be trayed, the first tower 102 could be packed while the second tower 104 is trayed, or the first tower 102 could be trayed while the second tower 104 is packed. Additionally, according to some embodiments, each tower 102, 104 could simultaneously include aspects of being packed and/or trayed. Each of the first and second towers 102, 104 can utilize chromatography to analyze the quality of the feed, intermediate product, and/or purge streams.

A packed tower/column is a term know in the art that can refer to a type of packed bed or other structure that includes packing material. In some embodiments a packed tower is preferred due to its low-pressure drop in vacuum operation, which reduces the pressure boost required from other equipment thus conserving energy and reducing wear on equipment. Packing material can be comprised of random small objects such as Raschig rings and/or packing material can be specifically designed. Packing material can also comprise adsorbents such as zeolite pellets, granular activated carbon, and the like. A packed tower can be a pressure structure that includes at least one packed portion/bed wherein the at least one packed portion/bed comprises packing material. A packed tower can include randomly packed material and/or can include structured packed sections which can be specifically arranged and/or stacked. Further, a packed tower can include liquid distributors and/or redistributors that help to distribute fluid evenly over packing material.

A trayed tower/column is a term known in the art that can refer to a tray/plate tower. A trayed tower can include one or more trays that are positioned on top of one another. A trayed tower can utilize reboiler(s) and condenser(s).

The system 100 can further include one or more feed vessels to hold and/or store crude ethanol. The crude ethanol can be pumped from the one or more crude ethanol drums and fed into other components of the system 100 via (for example) feed stream 101. According to some embodiments, the crude ethanol can be fuel ethanol and/or can originate from petrochemical and/or agricultural sources. The crude ethanol, such as fuel ethanol, can come from an ethanol plant such as a typical dry mill corn ethanol plant. According to some embodiments, the crude ethanol can be dry, crude ethanol. According to some embodiments, the crude ethanol stored in the one or more crude ethanol drums can be 200 proof and/or nearly 200 proof crude ethanol. However, the crude ethanol can contain any level of alcohol. According to some embodiments, the crude ethanol stored in the one or more crude ethanol vessels can be stored at 90 degrees Fahrenheit. However, the crude ethanol can be stored at any suitable temperature. The one or more crude ethanol vessels can be any sort of drum, vessel, container, and the like capable of holding and/or storing fluid.

The crude ethanol can be pumped, and/or otherwise transported, from the one or more crude ethanol vessels via a prefractionation feed pump 108 or drawn directly from the source facility into the first tower 102 by feed stream 101. The prefractionation feed pump 108 can be configured to move/circulate fluid, such as the crude ethanol, through various portion(s) of the system 100. For example, the prefractionation feed pump 108, according to some embodiments, is configured to move and/or circulate the crude ethanol from the one or more crude ethanol vessels to the first tower 102. The prefractionation feed pump 108 can be and/or comprise a peristaltic pump, a positive-displacement pump, a gear pump, a screw pump, a progressing cavity pump, a roots-type pump, a plunger pump, a rope pump, an impulse pump, a hydraulic pump, a velocity pump, a turbine pump, a gravity pump, a steam pump, a valveless pump, and/or any other suitable type of pump.

The first tower 102 can include a fluid entrance generally at or near a middle and/or center portion of the first tower 102 such that fluid, such as the crude ethanol, can enter a fluid entrance of the first tower 102 via crude ethanol feed 103 after being pumped through the prefractionation feed pump 108. Once crude ethanol has entered the first tower 102, the first tower 102 is configured to perform prefractionation to split heavy impurities and light impurities of the crude ethanol. This separation of heavy impurities and light impurities can create fractioned ethanol and/or fractioned portions of the ethanol. Heavy impurities in the crude ethanol can include heavier components based on boiling point, including, but not limited to, fusel alcohols such as 3-methyl butyl alcohol, normal propanol, and normal butanol; normal propanol is the lightest of the heavy impurities and can be a target to monitor for purposes of assessing appropriate removal of the heavy impurities. Light impurities in the crude ethanol can include, but are not limited to, methanol, acetaldehyde, acetal, and other light components of the crude ethanol; acetal is the heaviest of the light impurities and can be a target to monitor for purposes of assessing appropriate removal of light impurities. The heavy impurities and light impurities can be split, ttt and fluid comprising the heavy impurities and fluid comprising the light impurities can be sent to opposite ends of the second tower 104. Heavy impurities and/or fluid comprising heavy impurities can be referred to as “bottom” and/or “bottoms” herein. Light impurities and/or fluid comprising light impurities can be referred to as “top” and/or “tops” herein.

The prefractionation can be achieved via fractional distillation based on boiling point of various components of the crude ethanol.

According to some embodiments, a purpose of this prefractionation step, performed via the first tower 102 and/or other components, is to generally isolate a fraction of ethanol containing low-boiling point compounds from a fraction of ethanol containing higher boiling point compounds. This coarse split of light and heavy impurities makes a downstream finishing step (performed by the second tower 104 and/or other components) much more effective to concentrate and purge impurities while purifying the ethanol, thus allowing zero or near zero concentration for both heavy and light impurities in the final product in a more efficient manner.

According to some embodiments, a range of distillate-to-feed (D:F) ratios in the prefractionation step can range from 0.5 to 0.85 and can depend on: (1) the number of light versus heavy impurities in the crude ethanol and their concentrations, which can be predetermined by analysis; (2) the reflux-to-distillate (R:D) ratio of the prefractionation column, with a likely range of 2 to 5 based on mass; and (3) the actual performance of the distillation internals. According to various embodiments, other ranges of D:F ratios and other ranges of R:D ratios are contemplated herein. According to various embodiments, other HETP values and surface area-to-volume ratios for structured packing are contemplated herein.

Each of the first and second towers 102, 104 can internally include high density structured packing referenced above to maximize theoretical staging while reducing pressure drop.

According to some embodiments, the first tower 102 can include two packed beds, a first packed bed 110 and a second packed bed 112 wherein the first bed 110 is positioned above the second bed 112. While two packed beds are shown in the first tower 102, any number of packed beds can be used to further facilitate this process. Further, according to some embodiments, the first bed 110 can be approximately 6 theoretical equilibrium stages in length or height (such as units of feet or meters), and the second bed 112 depth can be approximately 12 theoretical stages. A center and/or middle portion of the first tower 102 where the crude ethanol is fed into the first tower 102 can be positioned between the first and second packed beds 110, 112 as shown in FIG. 1.

The first tower 102 can be configured so that it generates two exit streams wherein one exit stream exits the first tower 102 generally at or near a first end of the first tower 102 and the other exit stream exits the first tower 102 at a second end of the first tower 102. According to some embodiments, a distillate stream can exit from a top of the first tower 102 and a bottoms stream can exit from a bottom of the first tower 102. According to some embodiments, the distillate stream exits the top of the first tower 102 as a vapor and the bottoms stream exits the bottom of the first tower 102 as a liquid. The first tower may optionally include additional exit streams such as an optional vapor distillate line 109b in which vapor distillate that is immediately ready to be transferred may be transferred directly to the second tower 104.

According to some embodiments, as is shown in FIG. 1, a prefractionation condenser 114, a prefractionation reboiler or electrical heating element 118, and a prefractionation bottoms pump 122 (FIG. 1) can each be included as part of the system 100 and can each be operationally connected to the first tower 102. Common auxiliary equipment, such as reflux drums and pumps, stream traps, and control valves can also be included.

According to some embodiments, the distillate stream, which can comprise fluid comprising light impurities, is only partially condensed to generate liquid reflux 107 wherein the remaining vapor distillate 109 is sent to the second tower 104 in an effort to conserve energy. Further, a flow of cooling water 105 can be used to control the split of liquid reflux 107 and vapor distillate 109.

According to some embodiments, a fraction of the distillate exiting the top of the first tower 102 is sent to the second tower 104 (via optional vapor distillate line 109b), whereas a remaining portion, or balance, of distillate is routed to the prefractionation condenser 114. A valve can be positioned between the first tower 102 and the prefractionation condenser 114 on the stream exiting the top of the first tower 102 and entering the prefractionation condenser 114. This valve can control flow of fluid through this stream. In this scenario, a second valve would be helpful to control vapor distillate flow from the top of the first tower 102 to the second tower 104 via stream 109. One or more valves can be utilized to control fluid flow rate.

The prefractionation condenser 114 is operationally connected to the first tower 102 as shown in FIG. 1. The prefractionation condenser 114 can include a chilled water supply 105 (CWS) and a chilled water return (CWR); the CWS can include and/or provide cooling water from a cooling tower. The prefractionation condenser 114 can be any sort of condenser and can be used to cool liquids and/or to condense gaseous substances into a liquid state via cooling. According to some embodiments, the prefractionation condenser 114 can be a shell and tube condenser. At least some of the distillate (fluid comprising the light impurities) that exits the top of the first tower 102 can enter the prefractionation condenser 114 and be cooled and/or converted into a liquid wherein said liquid (also known as reflux) can be sent to a prefractionation reflux drum.

A prefractionation reflux drum will receive condensate from the condenser 114 and can be any sort of drum, vessel, container, and the like capable of holding and/or storing fluid. The prefractionation reflux drum is configured to store the reflux fluid wherein the reflux fluid 107 can then be sent back to the top of the first tower 102 where it can be reprocessed and sent to the second tower 104 as vapor or sent back to the prefractionation condenser 114. An inert bleed may be installed on the prefractionation reflux drum to provide non-condensable vapors and supplied with any suitable non-condensable gas.

A reflux fluid can be pumped out of prefractionation reflux drum and into the top of the first tower 102 via a prefractionation reflux pump. The prefractionation reflux pump may be configured to move and/or circulate the reflux fluid. The prefractionation reflux pump can be and/or comprise a peristaltic pump, a positive-displacement pump, a gear pump, a screw pump, a progressing cavity pump, a roots-type pump, a plunger pump, a rope pump, an impulse pump, a hydraulic pump, a velocity pump, a turbine pump, a gravity pump, a steam pump, a valveless pump, and/or any other suitable type of pump.

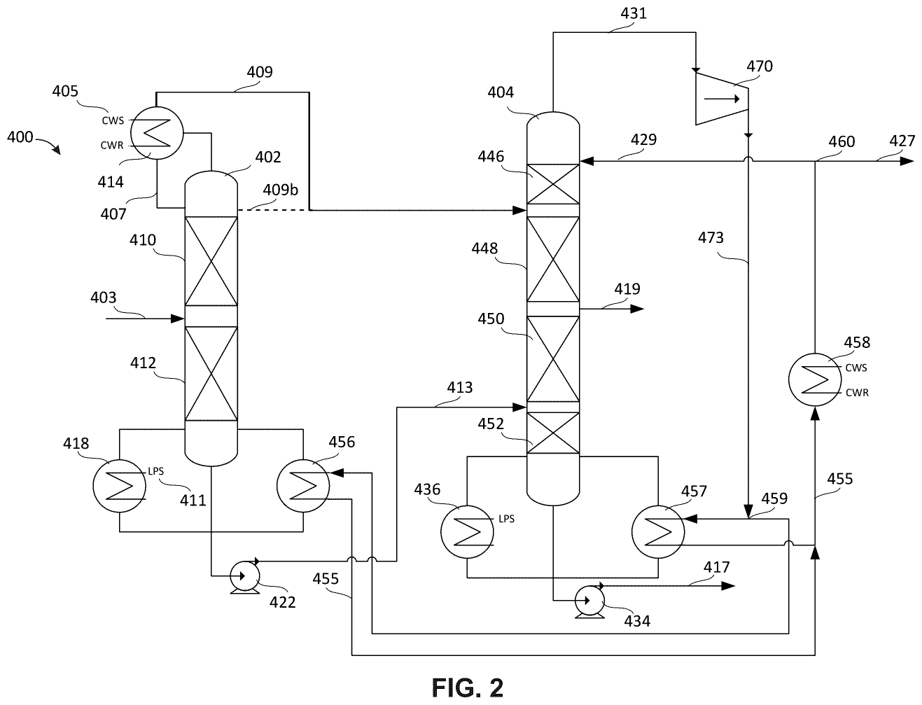

The system 100 further includes a prefractionation reboiler 118 that is operationally connected to the first tower 102 as shown in FIG. 1. The prefractionation reboiler 118 can be any sort of reboiler. The prefractionation reboiler 118 can also be configured with a stab-in heating element directly to a sump of the first tower 102. The prefractionation reboiler 118 can be configured to boil fluid comprising the heavy impurities of the crude ethanol located at the bottom of the first tower 102 and/or the prefractionation reboiler 118 can be configured to boil the fluid comprising the heavy impurities of the crude ethanol that exit the first tower 102. By boiling the fluid comprising heavy impurities of the crude ethanol, the prefractionation reboiler 118 can convert the fluid, which may be in liquid form, into vapor wherein said vapor re-enters the first tower 102 or is sent to the second tower 104. Fluid comprising the heavy impurities of the crude ethanol that exit the bottom of the first tower 102 (also known as bottoms) eventually enter a lower portion of the second tower 104 as shown in FIG. 1. According to some embodiments, the system 100 can optionally include the use of mechanical vapor recompression (MVR) as a means to drive the prefractionation reboiler 118 using overhead vapor, as shown in FIG. 2. The use of mechanical vapor recompression serves (MVR) as an energy recovery process and can lead to greater energy efficiency, cost-effectiveness, and environmental friendliness. The processes disclosed herein are not limited to mechanical vapor recompression.

As shown in FIG. 1, the prefractionation reboiler 118 can include a steam inlet 111 to facilitate boiling. This can be a recirculating reboiler, a thermosyphon reboiler, or a stab in heater. The system 100 can further include a prefractionation steam trap that is operationally connected to the prefractionation reboiler 118. The prefractionation steam trap can be any sort of steam trap. Such steam condensate pots, pads, and/or drums can be the same as and/or similar in nature to any other drum mentioned herein.

As shown in FIG. 1, the system further includes a prefractionation bottoms pump 122 that is operationally connected to the first tower 102, the second tower 104, and the prefractionation reboiler 118. The prefractionation bottoms pump 122 can be and/or comprise a peristaltic pump, a positive-displacement pump, a gear pump, a screw pump, a progressing cavity pump, a roots-type pump, a plunger pump, a rope pump, an impulse pump, a hydraulic pump, a velocity pump, a turbine pump, a gravity pump, a steam pump, a valveless pump, and/or any other suitable type of pump. The prefractionation bottoms pump 122 can be configured to pump, move, and/or circulate fluid between the first tower 102, the second tower 104, and/or the prefractionation reboiler 118. For example, the prefractionation bottoms pump 122 can be configured to move fluid comprising heavy impurities (also known as “bottoms”) from the first tower 102 to the second tower 104 and/or to the prefractionation reboiler 118.

According to some embodiments, the second tower 104 can have the same and/or similar characteristics as the first tower 102. Additionally, according to some embodiments, the second tower 104 can include the same and/or similar components as the first tower 102. According to some embodiments, the second tower 104 can be a packed tower and/or a trayed tower as described above. According to some embodiments, the second tower 104 can include four separate internal packed beds, wherein each of the packed beds are positioned in a same vertical plane on top of each other. According to some embodiments, the second tower includes four packed beds, a top bed 146 can be 2 to 4 theoretical stages in length or height (such as feet or meters), a second bed 148 from the top can be 15 to 20 theoretical stages, a third bed 150 from the top can be 10 to 14 theoretical stages, and a fourth bed 152 from the top can be 3 to 5 theoretical stages.

In some embodiments wherein the second tower includes four packed beds, the distillate stream comprising light impurities that exit the top end of the first tower 102 can enter the second tower 104 generally at a top/upper portion of the second tower 104. The distillate stream comprising the light impurities can enter the second tower 104 between the top packed bed 146 and the second bed 148 as shown in FIG. 1. The location at which the distillate stream comprising light impurities enters the second tower 104 allows for rectification of the light impurities while recovering purified ethanol.

In some embodiments wherein the second tower includes four packed beds, the bottoms stream comprising heavy impurities that exits the bottom end of the first tower 102 can enter the second tower 104 generally at a bottom/lower portion of the second tower 104. The bottoms stream comprising the heavy impurities can enter the second tower 104 between the fourth bed 152 (or bottom packed bed) and the third bed 150 as shown in FIG. 1. The location at which the bottoms stream comprising heavy impurities enters the second tower 104 allows for the stripping of purified ethanol while concentrating heavy impurities.

The distillate-to-feed (D:F) ratio of the second tower 104 can vary between 0.1 to 0.3 according to some embodiments. However, other D:F ratios are contemplated herein. The D:F ratio can be dependent on the types and concentrations of the impurities in the fuel ethanol. The reflux-to-distillate (R:D) ratio of the second tower 104 could be at or near 10:1, however, other ratios are contemplated herein. The R:D ratio of the second tower 104 is subject to change.

The second tower 104 can be configured such that the distillation of the separate light and heavy impurities originating in the first tower 102 is accomplished in a single finishing step in the second tower 104 wherein this final step actually functions as two distillation steps.

The second tower 104 can be configured to separate the light and heavy impurities from the ethanol to produce and/or capture a heart cut of purified ethanol. The second tower 104 is configured to send the heart cut to the middle/center portion of the second tower 104 wherein the heart cut purified ethanol can exit the second tower 104 at and/or near the middle/center portion (which can be referred to as the side draw) via an exit stream 119 as shown in FIG. 1 and described herein. According to embodiments in which the second tower 104 comprises four packing beds, the heart cut purified ethanol can exit the second tower 104 between the two middle packing beds, i.e., the second bed 148 and the third bed 150. Additionally, the second tower 104 can be configured to discard fluid comprising the light impurities and fluid comprising the heavy impurities from each end of the second tower 104 as is shown in FIG. 1 as well as described herein. The discarded fuel is still suitable for fuel ethanol and can be discarded from this system 100, but can be recaptured for fuel ethanol use.

As shown in FIG. 1, the system 100 further includes a finisher condenser 126, a finisher reflux pump 130, a finisher bottoms pump 134, a finisher reboiler 136, a finisher side draw pump 140, and optional polishing bed(s) 142.

The second tower 104 can be configured such that fluid comprising light impurities exit the second tower 104 at or near the top of the second tower 104. Such fluid can be sent from the second tower to the finisher condenser 126.

According to some embodiments, the finisher condenser 126 can be the same as or similar to the prefractionation condenser 114 and can be configured to operate in the same or similar manner as the prefractionation condenser 114. The finisher condenser 126 can be operationally connected to the second tower 104 as shown in FIG. 1. The finisher condenser 126 can include a chilled water supply 125 (CWS) and a chilled water return (CWR); the CWS can include and/or provide cooling water from a cooling tower. The finisher condenser 126 can be any sort of condenser and can be used to cool liquids and/or to condense gaseous substances into a liquid state via cooling. At least some, or all, of the fluid comprising the light impurities that exits the top of the second tower 104 can enter the finisher condenser 126 and be cooled and/or converted into a liquid wherein said liquid (also known as reflux) can be sent from the finisher condenser 126 to, for example, a finisher reflux drum.

The finisher reflux drum can be any sort of drum, vessel, container, and the like capable of holding and/or storing fluid. The finisher reflux drum is configured to store the reflux fluid wherein the reflux fluid can then be sent to one or more purge ethanol drums. The one or more purge ethanol drums can be any sort of drum, vessel, container, and the like capable of holding and/or storing fluid. The one or more purge ethanol drums can be configured to receive, hold, and/or store impure ethanol purged from the system 100.

The reflux fluid can be pumped out of finisher reflux drum and into the one or more purge ethanol drums via the finisher reflux pump 130. The finisher reflux pump 130 can be configured to pump, move, and/or circulate fluid. The finisher reflux pump 130 can be the same or similar to and/or can operate in the same or similar manner as the prefractionation reflux pump mentioned earlier. The finisher reflux pump 130 can be and/or comprise a peristaltic pump, a positive-displacement pump, a gear pump, a screw pump, a progressing cavity pump, a roots-type pump, a plunger pump, a rope pump, an impulse pump, a hydraulic pump, a velocity pump, a turbine pump, a gravity pump, a steam pump, a valveless pump, and/or any other suitable type of pump.

The second tower 104 can be configured such that fluid comprising the heavy impurities (also known as bottoms) exits the second tower 104 at and/or near the bottom of the second tower 104. Such fluid can be sent from the second tower 104 to one or more purge ethanol drums and/or to the finisher reboiler 136.

According to some embodiments, the finisher reboiler 136 can be the same as or similar to the prefractionation reboiler 118 and can operate in the same or similar manner as the prefractionation reboiler 118. The finisher reboiler 136 can be operationally connected to the second tower 104, the finisher bottoms pump 134, and one or more purge ethanol drums as shown in FIG. 1. The finisher reboiler 136 can be any sort of reboiler. The finisher reboiler 136 can be configured to boil the fluid comprising the heavy impurities of the ethanol located at and/or near the bottom of the second tower 104 and/or the finisher reboiler 136 can be configured to boil the fluid comprising the heavy impurities of the ethanol that exits the second tower 104. By boiling the heavy impurities of the ethanol, the finisher reboiler 136 can convert the heavy impurities, which may be in liquid form, into vapor wherein said vapor can re-enter the second tower 104 and/or can be sent to one or more purge ethanol drums. Fluid comprising heavy impurities of the ethanol that exits the bottom of the second tower 104 (also known as bottoms) can continue to cycle through the bottom of the second tower 104 until the heavy impurities are eventually sent to one or more purge ethanol drums via stream 117. According to some embodiments, the system 100 can optionally include the use of mechanical vapor recompression as a means to drive the finisher reboiler 136 using overhead vapor as shown in FIG. 2.

The finisher reboiler 136 can include a steam intake just as can be included with the prefractionation reboiler 118. The finisher reboiler 136 can be operationally connected to a steam trap. The finisher steam trap can be the same as or similar to the prefractionation steam trap and can operate in the same or similar manner as the prefractionation steam trap. The finisher steam trap can be any sort of steam trap. According to some embodiments, the finisher steam trap can function as an automatic valve that opens, closes, and/or modulates automatically. The finisher steam trap can be configured to discharge condensate, limit steam consumption, and/or discharge non-condensable fluids. The finisher steam trap can be and/or comprise a mechanical trap, a thermostatic trap, and/or a thermodynamic trap. Steam condensate from the finisher steam trap can be sent to one or more distillation pots, pads, and/or drums. Such steam condensate pots, pads, and/or drums can be the same as and/or similar in nature to any other drum mentioned herein.

The finisher bottoms pump 134 can be used to pump, move, and/or circulate fluid, such as fluid comprising the heavy impurities, from the second tower 104 and/or from the finisher reboiler 136 to the one or more purge ethanol drums. The finisher bottoms pump 134 can be the same as or similar to and/or can operate in the same or similar manner as any other pump mentioned herein. The finisher bottoms pump 134 can be and/or comprise a peristaltic pump, a positive-displacement pump, a gear pump, a screw pump, a progressing cavity pump, a roots-type pump, a plunger pump, a rope pump, an impulse pump, a hydraulic pump, a velocity pump, a turbine pump, a gravity pump, a steam pump, a valveless pump, and/or any other suitable type of pump.

As mentioned above, according to some embodiments, the purified ethanol can collect in a middle/central portion of the second tower 104. The purified ethanol can then exit the second tower 104 from the middle/central portion as shown in FIG. 1 via the exit stream 119. As such, in embodiments in which the second tower 104 includes four packed beds, the purified ethanol can exit the second tower 104 from an area located between the second and third beds 148, 150 from the middle of the second tower 104.

The purified ethanol can be pumped from the second tower 104 via the finisher side draw pump 140. The finisher side draw pump 140 can be the same as and/or similar to any pump described herein and can operate in the same and/or similar manner as any pump described herein. The finisher side draw pump 140 can be and/or comprise a peristaltic pump, a positive-displacement pump, a gear pump, a screw pump, a progressing cavity pump, a roots-type pump, a plunger pump, a rope pump, an impulse pump, a hydraulic pump, a velocity pump, a turbine pump, a gravity pump, a steam pump, a valveless pump, and/or any other suitable type of pump.

After the purified ethanol exits the second tower 104 via the exit stream 119, the purified ethanol can optionally be polished by running the purified ethanol over one or more activated carbon beds 142 or another suitable polishing technology to remove any trace of unpleasant odor or flavor. As shown in FIG. 1, the system 100 can include one (or more) polishing beds 142 according to some embodiments. However, any number of polishing beds could be included.

After the purified ethanol exits the second tower 104, and optionally the one or more polishing beds 142, the system 100 is configured such that the purified ethanol 123 can enter one or more high purity ethanol drums for storing or transport. In other words, one or more high purity ethanol drums can be configured to hold and/or store the purified ethanol produced by the system 100.

According to some embodiments, the two-tower approach and system 100 described herein could be accomplished by using a single column/tower with a dividing wall design wherein one vertical half of the column/tower would function as the prefractionation first tower 102 described above while the opposite half would function as the finishing second tower 104 as described above. This single-tower design is particularly suited for retrofits.

The system 100 can include one or more vacuums 124 to control and/or operate at least some aspects of the system 100. According to some embodiments, the one or more vacuums can utilize 2.5 to 5 PSIA, however, any suitable vacuum(s) and/or pressure rating could be used. A lower vacuum pressure provides the best separation; however, this can be limited by the power to the vacuum and the condenser's ability to provide sufficient cooling. By utilizing one or more vacuums, the impurity separation efficiency can be increased and/or improved while allowing distillation column/tower overhead condensation against typical cooling water. According to some embodiments, cooling water can be 75 degrees Fahrenheit, however, other temperatures could be used.

According to some embodiments, the system 100 can recover about 60% of the crude ethanol as purified ethanol. However, according to some embodiments, the system can recover more or less than 60% of the crude ethanol as purified ethanol. Additionally, the crude ethanol that is not recovered as and/or converted into purified ethanol can be used as fuel ethanol. Thus, the system 100 allows for essentially no yield loss of ethanol. The system 100 can include an outlet for the purge streams, wherein the purged ethanol and/or heavy and/or light impurities can be used in the fuel ethanol industry. For example, the purged ethanol and/or heavy and/or light impurities can be used as a fuel additive. By being able to use the purged ethanol and/or heavy and/or light impurities in the fuel industry as they are without modification, the need to sell the purge streams to other fuel ethanol producers or gasoline blenders/distributors is eliminated. Thus, the system 100 minimizes and/or eliminates loss of value of the ethanol and leads to economic efficiency, energy efficiency, and environmental friendliness.

The system 100 is capable of drastically reducing the concentration of impurities in ethanol. For example, the system 100 is capable of receiving crude ethanol that comprises impurities ranging from 1000-2000 ppm and reducing those total impurities down to below 10 ppm and in some cases below detection limit (<0.1 ppm). While the reduction of impurities from 1000-2000 ppm to <10 ppm is provided as an example, the system 100 is capable of reducing other ranges of impurities. For example, the system could receive crude ethanol having impurities greater than 2000 ppm and reduce such impurities to less than 10 ppm. According to some embodiments, the system 100 is capable of reducing the amount of methanol, and/or other impurities, to less than 10 grams per liter of ethanol produced by the system 100.

The system 100, and components thereof such as the one or more polishing beds 142, is configured to minimize and/or eliminate any trace of unpleasant odor or flavor in the ethanol processed by the system 100.

When the present disclosure refers to a “purified”, “refined”, and/or “distilled” chemical, such as ethanol, such purified, refined, and/or distilled chemical is the chemical that has been processed and output by the system 100 and/or the system 400. According to some embodiments, said purified, refined, and/or distilled chemical that is output by the system 100 and/or the system 400 is essentially absolutely pure. According to some embodiments, said purified, refined, and/or distilled chemical that is output by the system 100 and/or the system 400 has impurity levels that are less than about 400 ppm, less than about 350 ppm, less than about 300 ppm, less than about 250 ppm, less than about 200 ppm, less than about 180 ppm, less than about 160 ppm, less than about 140 ppm, less than about 120 ppm, less than about 100 ppm, less than about 90 ppm, less than about 80 ppm, less than about 70 ppm, less than about 60 ppm, less than about 50 ppm, less than about 40 ppm, less than about 30 ppm, less than about 20 ppm, or less than about 10 ppm. According to some embodiments, said purified, refined, and/or distilled chemical that is output by the system 100 and/or the system 400 has impurity levels that are suitable to be used in a particular industry such as the food and beverage industry, the pharmaceutical industry, and other laboratory industries. Laboratory industry use of ethanol includes, but is not limited to, use of ethanol as a solvent in chemical reactions, use of ethanol for cleaning and sterilizing lab equipment, and use of ethanol for preserving biological samples. According to some embodiments, said purified, refined, and/or distilled chemical that is output by the system 100 and/or the system 400 has impurity levels less than 10 grams per liter of ethanol.

According to some embodiments, prior to the crude ethanol entering the first tower 102, the system 100, and/or method(s), algorithm(s), and/or other aspects described herein, is configured to add a small amount of water in certain circumstances which may be beneficial to enhancing the volatility of some difficult-to-remove impurities (valeraldehye, [C₅H₁₀O] as an example).

According to some embodiments, the benefit of adding water to the crude ethanol fed into the first tower 102 and/or adding water to the crude ethanol entrance of the first tower 102 can be understood only after the impurity types and their concentrations are analyzed and evaluated by the system 100, and/or method(s), algorithm(s), or other aspects described herein, with optimization capability.

According to some embodiments, the system 100, and/or method(s), algorithm(s), and/or other aspects described herein, is set up to simultaneously vary water addition, the first tower 102 D:F ratio, and the second tower 104 D:F ratio to maximize impurity removal from ethanol and/or to maximize the purified ethanol yield while meeting concentration specifications of key impurities.

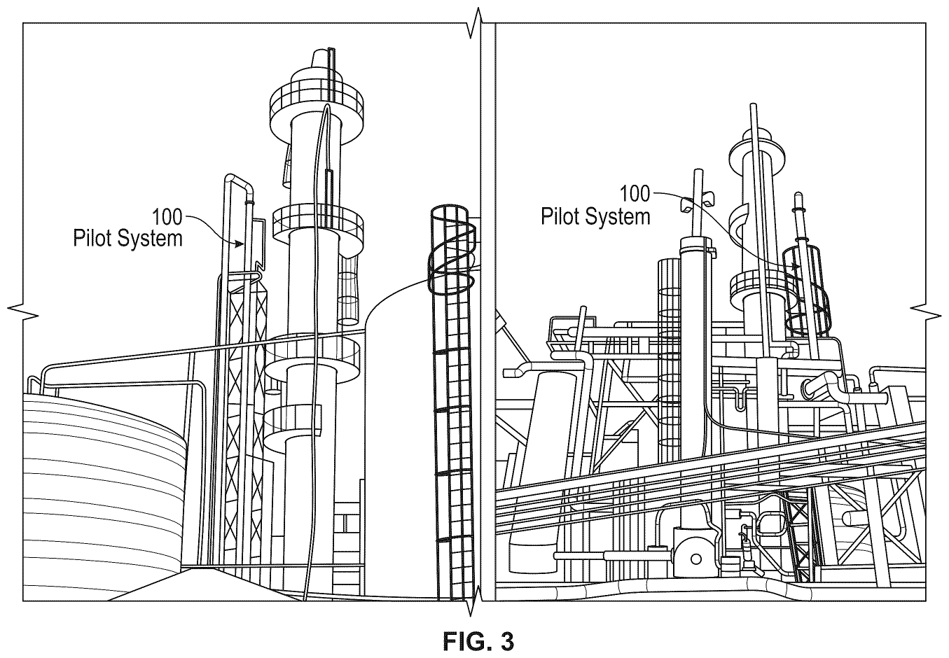

FIG. 3 shows two perspective views of at least some components of the system 100, wherein said component(s) of the system 100 are shown to be configured for use of the system 100.

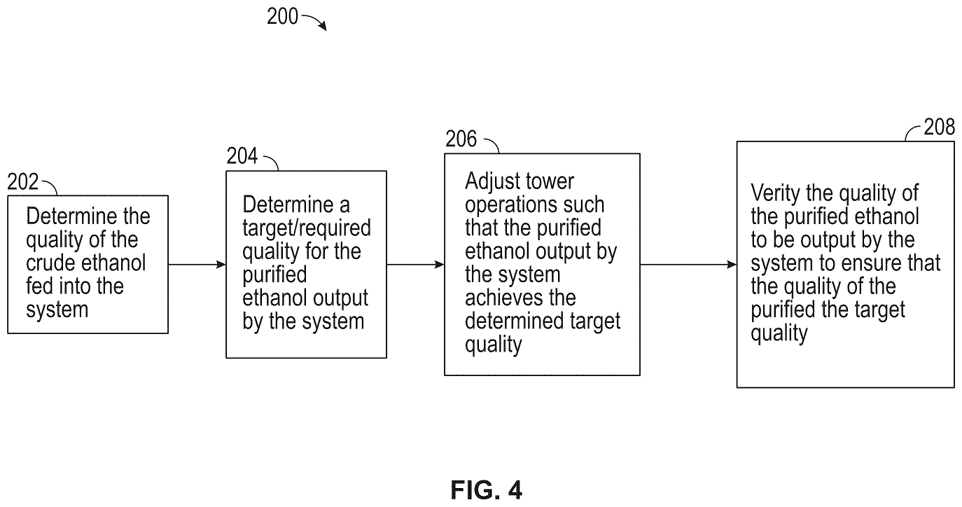

FIG. 4 shows a flow diagram of an algorithm 200 wherein said algorithm 200 can be used to control and/or operate the system 100 and/or the system 400. According to some embodiments, the algorithm 200 can be a feedforward control. According to some embodiments, the algorithm 200 can be a feedback control. According to some embodiments, the algorithm 200 can incorporate characteristics of both feedforward and feedback controls. Typically, the feedback control can analyze the product and provide information back to the controller and/or user such that adjustments can be made to tailor the product. The feedforward control can analyze the gas or liquid feed (e.g., to assess the makeup of heavy and light impurities) so that adjustments can be made to remove targeted impurities. This analysis is performed via an analytical instrument as discussed further below.

A feedforward algorithm and/or process control involves control action independent of any output or variables. A feedforward algorithm operates in a pre-determined manner and does not deviate from the pre-determined operation or react to any sort of change in circumstance. A feedback algorithm and/or process control involves using feedback to control aspects of a system. Feedback could include metrics, inputs, outputs, and/or other factors that could affect the operation of the system 100 and/or the system 400. A feedback feedforward control can utilize a feedforward control to utilize the pre-determined operations and use a feedback control to respond to feedback in the system such as disturbances, modeling, variations from a set point, and measurement errors.

The algorithm 200 used to control and/or operate the system 100 and/or the system 400 can include a step 202 to measure, analyze, and/or determine the quality of the crude ethanol fed into the system 100 from one or more crude ethanol drums and/or fed into the system 400 from the feed 406. Measuring, analyzing, and/or determining the quality of crude ethanol fed into the system 100 and/or the system 400 can be performed via gas chromatography. Gas chromatography is a type of chromatography used in analytical chemistry for separating and analyzing compounds that can be vaporized without decomposition. According to some embodiments, determining and/or measuring the quality of the crude ethanol can be performed via liquid chromatography (including, but not limited to, high-performance liquid chromatography (HPLC), ultra-performance liquid chromatography (UPLC), etc.). The system 100 and/or the system 400 can include whatever sensors, analytical equipment, and/or any other component(s) necessary to perform chromatography.

Measuring, analyzing, and/or determining the quality of the crude ethanol fed into the system 100 and/or the system 400 can include, but is not limited to, testing, measuring, identifying, and/or determining the purity of the crude ethanol, the types, categories, species, and quantities of impurities of the crude ethanol, the boundaries in terms of percentages/concentrations of those impurities, the moisture content of the crude ethanol, and the acidity of the crude ethanol.

The algorithm 200 can further include a step 204 of determining a target/required quality for the purified ethanol. The target quality for the purified ethanol can be determined automatically by the system 100, the system 400, and/or by the algorithm 200 based on the industry and/or product in which the purified ethanol will be used. According to some embodiments, the target quality for the purified ethanol can be determined based on user input. For example, a user can enter a particular target quality which the purified ethanol must reach by the time the purified ethanol is released from the system 100 and/or the system 400. Just as for the quality of the crude ethanol, the target quality can include, but is not limited to, data such as purity for the purified ethanol, the types, categories, species, and quantities of impurities for the purified ethanol, the boundaries in terms of percentages/concentrations of those impurities, the moisture content for the purified ethanol, and the acidity for the purified ethanol.

The algorithm 200 can further include another step 206 of adjusting the tower operations, as well as any other aspects of the system 100 and/or the system 400, such that the purified ethanol produced by the system achieves the determined target quality. Such adjustable tower operations can include, but are not limited to, adjusting the depth of vacuum, adjusting the distillate-to-feed (D:F) ratio, adjusting the reflux-to-distillate (R:D) ratio, adjusting the addition of water to the crude ethanol fed into the first tower 102 and/or the prefractionation tower 402 (of FIG. 2), adjusting the bottoms rate, and any other operations of the system 100 and/or the system 400 that could be adjusted in order for the purified ethanol to achieve the target quality. The tower operations, controls, and/or other operations of the system 100 and/or the system 400 can be adjusted and/or tuned to produce a range of ethanol purities. The adjustments needed to produce a range of ethanol purities can depend on the quality of the crude ethanol fed into the system 100 and/or the system 400 as well as the desired industry and/or product and the target quality for the purified ethanol produced by the system 100 and/or the system 400.

According to some embodiments, the side draw rate can be adjusted to purge either the heavy impurities or light impurities; this is particularly helpful when there are larger volumes of impurities either on the heavy or light side. According to some embodiments, the side draw rate can be adjusted so as to remove heavy impurities, then adjusted to remove light impurities, or vice versa. If there are minimal impurities remaining, it may be more suitable to raise the reflux rate.

The algorithm 200 can further include a step 208 of measuring, analyzing, and/or verifying the actual quality of the purified ethanol to ensure that it meets the target quality. This measurement, analysis, and/or verification can be performed via gas chromatography and/or liquid chromatography. Just as for the quality of the crude ethanol, the quality of the purified ethanol can include, but is not limited to, data such as purity of the purified ethanol, the types, categories, species, and quantities of impurities of the purified ethanol, the boundaries in terms of percentages/concentrations of these impurities, the moisture content of the purified ethanol, and the acidity of the purified ethanol.

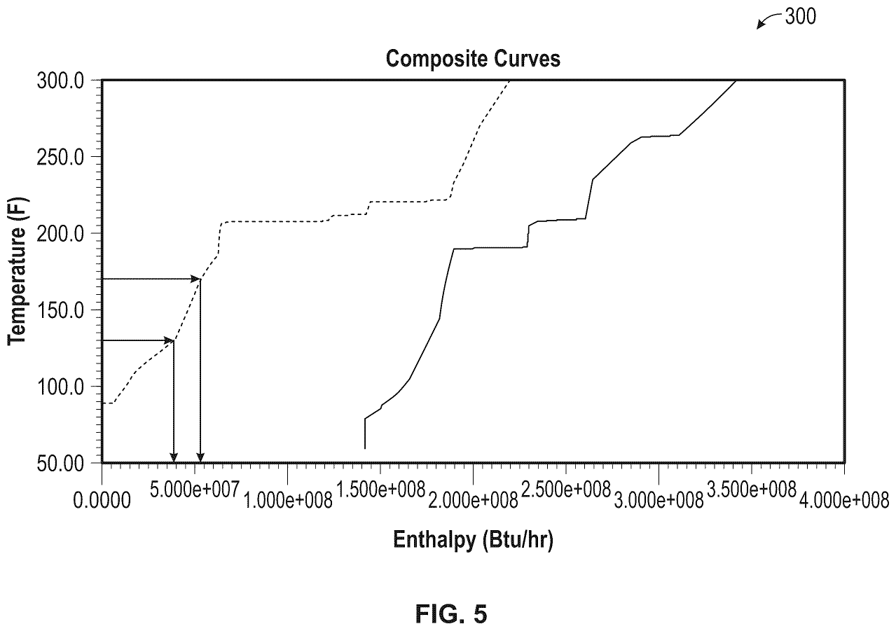

FIG. 5 shows a graphical representation 300 of composite curves of enthalpy versus temperature related to the system 100. The high purity ethanol process of the system 100 allows for some fraction of a dry mill ethanol plant total product. The high purity ethanol combined with a relatively low boiling point in the high purity ethanol towers 102, 104 (nearly pure ethanol distilled at 3 psia) provides the opportunity to integrate the high purity distillation with multiple possible streams in the main process. The graphical representation 300 of FIG. 5 shows enthalpy versus temperature for a 50 MGY (millions of gallons per year) ethanol plant applying the system 100. The dotted line of the graphical representation 300 shows the expected available duty of “hot streams” (a stream that requires cooling in the process). As shown in the graphical representation 300 of FIG. 5, based on a pinch analysis, the expected available duty of hot streams between the temperatures of about 130 degrees Fahrenheit to about 160 degrees Fahrenheit provides a source of heating for the about 115 degree Fahrenheit to about 120 degree Fahrenheit boiling environment in the high purity towers 102, 104. As shown in the graphical representation 300 of FIG. 5, up to about 15 MMBTU/hr (Metric Million British Thermal Unit/hour) is available between about 130 degrees Fahrenheit and about 160 degrees Fahrenheit for a 50 MGY sized plant.

FIG. 2 shows an example embodiment of a chemical purification, distillation and/or refinement system and/or assembly 400 according to aspects of the present disclosure. While the schematic shown in FIG. 2 is generally referred to as the system 400 herein, it could also be referred to as an assembly 400. Further, while much of the present disclosure focuses on the purification of ethanol, the system 400 could be used to purify, distill, and/or refine any suitable chemical. Again, whenever the present disclosure refers to “ethanol”, any suitable chemical could be substituted. It should be noted that the arrows appearing on any lines, piping, hoses, ducts, streams, channels, and/or any other passages in FIG. 2 represent the flow of fluid through said lines, piping, hoses, ducts, streams, channels, and/or passages.

The system 400 can be the same as and/or similar to the system 100 with the addition of various components. As such, the system 400 can function in a similar manner as the system 100. The system 400 can include and/or embody any characteristic(s) and/or aspect(s) of the system 100. Component(s) of the system 400 that are generally the same as and/or similar to component(s) of the system 100 can perform generally the same task and/or can function in generally the same manner as those component(s) of the system 100.

The system 400 can be used for mechanical vapor recompression (MVR). MVR can comprise an energy recovery process which can be used to recycle waste heat to improve efficiency. As shown in FIG. 2, the system 400 mirrors the basic concept of FIG. 1, system 100, where a crude ethanol feed 103 is fed to a prefractionation tower 102 to generate an overhead light cut 109 and bottom-heavy cut 113. The heavy and light cuts are fed to the finishing tower 104, as described above with relation to system 100, and fractionated into an overhead light purge 127, a side product draw 119, and a bottom-heavy purge 117. The towers will be similarly equipped with a prefractionation condenser and reflux equipment 114, bottom steam reboilers, 118 and 136, and may include all other equipment described for system 100.

According to some embodiments, the first tower 402 can include two packed beds, a first packed bed 410 and a second packed bed 412 wherein the first bed 410 is positioned above the second bed 412. While two packed beds are shown in the first tower 402, any number of packed beds can be used to further facilitate this process. Further, according to some embodiments, the first bed 410 can be approximately 6 theoretical equilibrium stages in length or height, and the second bed 412 depth can be approximately 12 theoretical stages. A center and/or middle portion of the first tower 402 where the crude ethanol is fed into the first tower 402 can be positioned between the first and second packed beds 410, 412 as shown in FIG. 2.

The first tower 402 can be configured so that it generates two exit streams wherein one exit stream exits the first tower 402 generally at or near a first end of the first tower 402 and the other exit stream exits the first tower 402 at a second end of the first tower 402. According to some embodiments, a distillate stream can exit from a top of the first tower 402 and a bottoms stream can exit from a bottom of the first tower 402. According to some embodiments, the distillate stream exits the top of the first tower 402 as a vapor and the bottoms stream exits the bottom of the first tower 402 as a liquid. The first tower may optionally include additional exit streams such as an optional vapor distillate line 409b in which vapor distillate that is immediately ready to be transferred may be transferred directly to the second tower 404. Further, the system 400 includes an exit stream directed towards MVR reboiler 456. More will be discussed on the MVR reboiler 456 with its relation to other components in system 400.

According to some embodiments, as is shown in FIG. 1, a prefractionation condenser 414, a prefractionation reboiler or electrical heating element 418, and a prefractionation bottoms pump 422 (FIG. 2) can each be included as part of the system 400 and can each be operationally connected to the first tower 402. Common auxiliary equipment, such as reflux drums and pumps, stream traps, and control valves can also be included.

According to some embodiments, the distillate stream, which can comprise fluid comprising light impurities, is only partially condensed to generate liquid reflux 407 wherein the remaining vapor distillate 409 is sent to the second tower 404 in an effort to conserve energy. Further, a flow of cooling water 405 can be used to control the split of liquid reflux 407 and vapor distillate 409.

According to some embodiments, a fraction of the distillate exiting the top of the first tower 402 is sent to the second tower 404 (via optional vapor distillate line 409b), whereas a remaining portion, or balance, of distillate is routed to the prefractionation condenser 414. A valve can be positioned between the first tower 402 and the prefractionation condenser 414 on the stream exiting the top of the first tower 402 and entering the prefractionation condenser 414. This valve can control flow of fluid through this stream. In this scenario, a second valve would be helpful to control vapor distillate flow from the top of the first tower 402 to the second tower 404 via stream 409. One or more valves can be utilized to control fluid flow rate.

The prefractionation condenser 414 is operationally connected to the first tower 402 as shown in FIG. 2. The prefractionation condenser 414 can include a chilled water supply 405 (CWS) and a chilled water return (CWR); the CWS can include and/or provide cooling water from a cooling tower. The prefractionation condenser 414 can be any sort of condenser and can be used to cool liquids and/or to condense gaseous substances into a liquid state via cooling. According to some embodiments, the prefractionation condenser 414 can be a shell and tube condenser. At least some of the distillate (fluid comprising the light impurities) that exits the top of the first tower 402 can enter the prefractionation condenser 414 and be cooled and/or converted into a liquid wherein said liquid (also known as reflux) can be sent to a prefractionation reflux drum.

A prefractionation reflux drum will receive condensate from the condenser 414 and can be any sort of drum, vessel, container, and the like capable of holding and/or storing fluid. The prefractionation reflux drum is configured to store the reflux fluid wherein the reflux fluid 407 can then be sent back to the top of the first tower 402 where it can be reprocessed and sent to the second tower 404 as vapor or sent back to the prefractionation condenser 414. An inert bleed may be installed on the prefractionation reflux drum to provide non-condensable vapors and supplied with any suitable non-condensable gas.

A reflux fluid can be pumped out of prefractionation reflux drum and into the top of the first tower 402 via a prefractionation reflux pump. The prefractionation reflux pump may be configured to move and/or circulate the reflux fluid. The prefractionation reflux pump can be and/or comprise a peristaltic pump, a positive-displacement pump, a gear pump, a screw pump, a progressing cavity pump, a roots-type pump, a plunger pump, a rope pump, an impulse pump, a hydraulic pump, a velocity pump, a turbine pump, a gravity pump, a steam pump, a valveless pump, and/or any other suitable type of pump.

The system 400 further includes a prefractionation reboiler 418 that is operationally connected to the first tower 402 as shown in FIG. 2. The prefractionation reboiler 418 can be any sort of reboiler. The prefractionation reboiler 418 can also be configured with a stab-in heating element directly to a sump of the first tower 402. The prefractionation reboiler 418 can be configured to boil fluid comprising the heavy impurities of the crude ethanol located at the bottom of the first tower 402 and/or the prefractionation reboiler 418 can be configured to boil the fluid comprising the heavy impurities of the crude ethanol that exit the first tower 402. By boiling the fluid comprising heavy impurities of the crude ethanol, the prefractionation reboiler 418 can convert the fluid, which may be in liquid form, into vapor wherein said vapor re-enters the first tower 402 or is sent to the second tower 404. Fluid comprising the heavy impurities of the crude ethanol that exit the bottom of the first tower 402 (also known as bottoms) eventually enter a lower portion of the second tower 404 as shown in FIG. 2. As shown in FIG. 2, the system 400 can includes the use of mechanical vapor recompression (MVR) as a means to drive the prefractionation reboiler 418 using overhead vapor. The use of mechanical vapor recompression serves (MVR) as an energy recovery process and can lead to greater energy efficiency, cost-effectiveness, and environmental friendliness. The processes disclosed herein are not limited to mechanical vapor recompression.

As shown in FIG. 2, the prefractionation reboiler 418 can include a steam inlet 411 to facilitate boiling. This can be a recirculating reboiler, a thermosyphon reboiler, or a stab in heater. The system 400 can further include a prefractionation steam trap that is operationally connected to the prefractionation reboiler 418. The prefractionation steam trap can be any sort of steam trap. Such steam condensate pots, pads, and/or drums can be the same as and/or similar in nature to any other drum mentioned herein.

As shown in FIG. 2, the system further includes a prefractionation bottoms pump 422 that is operationally connected to the first tower 402, the second tower 404, and the prefractionation reboiler 418. The prefractionation bottoms pump 422 can be and/or comprise a peristaltic pump, a positive-displacement pump, a gear pump, a screw pump, a progressing cavity pump, a roots-type pump, a plunger pump, a rope pump, an impulse pump, a hydraulic pump, a velocity pump, a turbine pump, a gravity pump, a steam pump, a valveless pump, and/or any other suitable type of pump. The prefractionation bottoms pump 422 can be configured to pump, move, and/or circulate fluid between the first tower 402, the second tower 404, and/or the prefractionation reboiler 418. For example, the prefractionation bottoms pump 422 can be configured to move fluid comprising heavy impurities (also known as “bottoms”) from the first tower 402 to the second tower 404 and/or to the prefractionation reboiler 418.

According to some embodiments, the second tower 404 can have the same and/or similar characteristics as the first tower 402. Additionally, according to some embodiments, the second tower 404 can include the same and/or similar components as the first tower 402. According to some embodiments, the second tower 404 can be a packed tower and/or a trayed tower as described above. According to some embodiments, the second tower 404 can include four separate internal packed beds, wherein each of the packed beds are positioned in a same vertical plane on top of each other. According to some embodiments, the second tower includes four packed beds, a top bed 446 can be 2 to 4 theoretical stages in length or height (such as feet or meters), a second bed 448 from the top can be 15 to 20 theoretical stages, a third bed 450 from the top can be 10 to 14 theoretical stages, and a fourth bed 452 from the top can be 3 to 5 theoretical stages.

In some embodiments wherein the second tower includes four packed beds, the distillate stream comprising light impurities that exit the top end of the first tower 402 can enter the second tower 404 generally at a top/upper portion of the second tower 404. The distillate stream comprising the light impurities can enter the second tower 404 between the top packed bed 446 and the second bed 448 as shown in FIG. 2. The location at which the distillate stream comprising light impurities enters the second tower 404 allows for rectification of the light impurities while recovering purified ethanol.

In some embodiments wherein the second tower includes four packed beds, the bottoms stream comprising heavy impurities that exits the bottom end of the first tower 402 can enter the second tower 404 generally at a bottom/lower portion of the second tower 404. The bottoms stream comprising the heavy impurities can enter the second tower 404 between the fourth bed 452 (or bottom packed bed) and the third bed 450 as shown in FIG. 1. The location at which the bottoms stream comprising heavy impurities enters the second tower 404 allows for the stripping of purified ethanol while concentrating heavy impurities.

The distillate-to-feed (D:F) ratio of the second tower 404 can vary between 0.1 to 0.3 according to some embodiments. However, other D:F ratios are contemplated herein. The D:F ratio can be dependent on the types and concentrations of the impurities in the fuel ethanol. The reflux-to-distillate (R:D) ratio of the second tower 404 could be at or near 10:1, however, other ratios are contemplated herein. The R:D ratio of the second tower 404 is subject to change.

The second tower 404 can be configured such that the distillation of the separate light and heavy impurities originating in the first tower 402 is accomplished in a single finishing step in the second tower 404 wherein this final step actually functions as two distillation steps.

The second tower 404 can be configured to separate the light and heavy impurities from the ethanol to produce and/or capture a heart cut of purified ethanol. The second tower 404 is configured to send the heart cut to the middle/center portion of the second tower 404 wherein the heart cut purified ethanol can exit the second tower 404 at and/or near the middle/center portion (which can be referred to as the side draw) via an exit stream 419 as shown in FIG. 2 and described herein. According to embodiments in which the second tower 404 comprises four packing beds, the heart cut purified ethanol can exit the second tower 404 between the two middle packing beds, i.e., the second bed 448 and the third bed 450. Additionally, the second tower 404 can be configured to discard fluid comprising the light impurities and fluid comprising the heavy impurities from each end of the second tower 404 as is shown in FIG. 1 as well as described herein. The discarded fuel is still suitable for fuel ethanol and can be discarded from this system 400, but can be recaptured for fuel ethanol use.

The second tower 404 can be configured such that fluid comprising light impurities exit the second tower 404 at or near the top of the second tower 404. Such fluid can be sent from the second tower to the compressor 470. The system 400 utilizes a compressor 470 to compress finishing column overhead vapors 431 to an elevated pressure above the operating conditions of first and second towers 402 and 404. The pressure increase may range from 2 to 8 psi, or in some embodiments range 4 to 6 psi. In essence, the compression step will superheat the light vapors, which can then act as steam for auxiliary reboilers 456 and 457, which then assist prefractionation reboiler 418 and finisher reboiler 436 to improve efficiency of the system 400. The compressor 470 can be any equipment suitable to accomplish the desired level of compression, such as a single or multi-stage centrifugal compressor, or single or multi-stage blower or fan.

According to some embodiments, reboiler surface areas for 418 and 436 may be reduced to compensate for the additional reboilers 456 and 457. The heat exchanger design aspects should consider any previously mentioned definition and include any equipment design that accomplishes the goal of receiving column bottom liquid, boiling such liquid, and returning it into the column as vapor. The reboilers can be designed as recirculating reboilers, thermosyphon reboilers, or stab in heaters, and not be limited to general type such as shell and tube or plate and frame.

According to some embodiments, the split of superheated ethanol vapor between 456 and 457 can be controlled at a process control variable 459. Any suitable valving manifold can be used for the process control variable 459 and should allow flexibility to target flow ranges of 20%-80% to reboiler 457. Other split ranges may target 40%-60%. Alternatively, the process may be designed to run only one MVR reboiler, 456 or 457, in which case the split variable is non-existent.

According to some embodiments, reboilers 456 and 457 will condense hot ethanol vapor. The combined condensed ethanol 455 will enter a trim cooler 458 to produce cooled light ends purge 427 and finishing column reflux 429. The cooler 458 can be supplied with chilled cooling water, or alternative cooling fluid, capable of bringing the reflux to 80 degrees F. In some embodiments the trim cooler 458 may cool fluid 455 to a range of 90-100 degrees F. The extent of cooling ultimately needs to reflect the depth of vacuum for which the top of tower 404 is run. The trim cooler may be designed as, but not limited to, shell and tube or plate and frame heat exchangers.

The second tower 404 can be configured such that fluid comprising the heavy impurities (also known as bottoms) exits the second tower 404 at and/or near the bottom of the second tower 404. Such fluid can be sent from the second tower 404 to one or more purge ethanol drums and/or to the finisher reboiler 436.

According to some embodiments, the finisher reboiler 436 can be the same as or similar to the prefractionation reboiler 418 and can operate in the same or similar manner as the prefractionation reboiler 418. The finisher reboiler 436 can be operationally connected to the second tower 404, the finisher bottoms pump 434, and one or more purge ethanol drums as shown in FIG. 2. The finisher reboiler 436 can be any sort of reboiler. The finisher reboiler 436 can be configured to boil the fluid comprising the heavy impurities of the ethanol located at and/or near the bottom of the second tower 404 and/or the finisher reboiler 436 can be configured to boil the fluid comprising the heavy impurities of the ethanol that exits the second tower 404. By boiling the heavy impurities of the ethanol, the finisher reboiler 436 can convert the heavy impurities, which may be in liquid form, into vapor wherein said vapor can re-enter the second tower 404 and/or can be sent to one or more purge ethanol drums. Fluid comprising heavy impurities of the ethanol that exits the bottom of the second tower 404 (also known as bottoms) can continue to cycle through the bottom of the second tower 404 until the heavy impurities are eventually sent to one or more purge ethanol drums via stream 417. According to some embodiments, the system 400 can optionally include the use of mechanical vapor recompression as a means to drive the finisher reboiler 436 using overhead vapor as shown in FIG. 2.

The finisher reboiler 436 can include a steam intake just as can be included with the prefractionation reboiler 418. The finisher reboiler 436 can be operationally connected to a steam trap. The finisher steam trap can be the same as or similar to the prefractionation steam trap and can operate in the same or similar manner as the prefractionation steam trap. The finisher steam trap can be any sort of steam trap. According to some embodiments, the finisher steam trap can function as an automatic valve that opens, closes, and/or modulates automatically. The finisher steam trap can be configured to discharge condensate, limit steam consumption, and/or discharge non-condensable fluids. The finisher steam trap can be and/or comprise a mechanical trap, a thermostatic trap, and/or a thermodynamic trap. Steam condensate from the finisher steam trap can be sent to one or more distillation pots, pads, and/or drums. Such steam condensate pots, pads, and/or drums can be the same as and/or similar in nature to any other drum mentioned herein.

The finisher bottoms pump 434 can be used to pump, move, and/or circulate fluid, such as fluid comprising the heavy impurities, from the second tower 404 and/or from the finisher reboiler 436 to the one or more purge ethanol drums. The finisher bottoms pump 434 can be the same as or similar to and/or can operate in the same or similar manner as any other pump mentioned herein. The finisher bottoms pump 434 can be and/or comprise a peristaltic pump, a positive-displacement pump, a gear pump, a screw pump, a progressing cavity pump, a roots-type pump, a plunger pump, a rope pump, an impulse pump, a hydraulic pump, a velocity pump, a turbine pump, a gravity pump, a steam pump, a valveless pump, and/or any other suitable type of pump.

As mentioned above, according to some embodiments, the purified ethanol can collect in a middle/central portion of the second tower 404. The purified ethanol can then exit the second tower 404 from the middle/central portion as shown in FIG. 1 via the exit stream 419. As such, in embodiments in which the second tower 404 includes four packed beds, the purified ethanol can exit the second tower 404 from an area located between the second and third beds 448, 450 from the middle of the second tower 404.

According to some embodiments, the two-tower approach and system 400 described herein could be accomplished by using a single column/tower with a dividing wall design wherein one vertical half of the column/tower would function as the prefractionation first tower 402 described above while the opposite half would function as the finishing second tower 404 as described above. This single-tower design is particularly suited for retrofits.

According to some embodiments, the system 400 includes a flow control manifold 460 to split subcooled ethanol between finishing column reflux 429 and cooled light ends purge 427. The split of subcooled ethanol between finishing column reflux 429 and cooled light ends purge 427 should be considered a design parameter, where a reflux ratio can be controlled. It may be necessary to increase/decrease this reflux ratio after receiving analytical feedforward information from the crude ethanol or feedback information from the products and purges. Any suitable valving or flow control manifold can be used for the flow control manifold 460.

As noted, the system 400 shown in FIG. 2 allows for the use of MVR. According to some embodiments, the total energy consumed by the system 400 is 7,700 to 9,300 BTU/gallon of product produced, as opposed to the total energy (as power) consumed by the system 100 being 16,000 to 19,000 BTU/gallon of product produced. The energy consumed by the system 100 and/or system 400 is significantly lower than ethanol purification systems in the prior art. Thus, the system 100 and the system 400 are both more energy efficient and environmentally friendly than ethanol purification systems in the prior art.

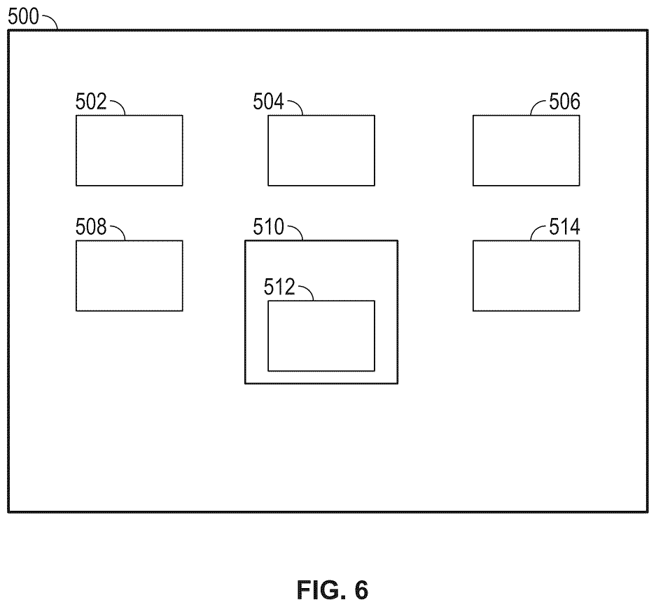

The system 100 and/or the system 400 can include a cyberinfrastructure 500, as shown in FIG. 6, wherein the cyberinfrastructure 500 can include software, firmware, and/or additional hardware to implement the algorithm 200 and/or control/operate functionality of the system 100 and/or the system 400. According to some embodiments, the methods and/or algorithm(s) described herein, including the algorithm 200, can be implemented using programmatic modules, engines, and/or components. A programmatic module, engine, or component can include a program, a sub-routine, a portion of a program, or a software component or a hardware component capable of performing one or more stated tasks or functions. A module or component can exist on a hardware component independently of other modules or components. Alternatively, a module or component can be a shared element or process of other modules, programs, or machines.

Dedicated hardware implementations including, but not limited to, application specific integrated circuits, programmable logic arrays, and/or other hardware devices can likewise be constructed to implement the methods and/or algorithm(s) described herein. Applications that may include the apparatus and systems of various embodiments broadly include a variety of electronic and computer systems. Some embodiments implement functions in two or more specific interconnected hardware modules, or devices, with related control and data signals communicated between and through the modules, or as portions of an application-specific integrated circuit. Thus, the example system is applicable to software, firmware, and hardware implementations.

In accordance with various embodiments of the present disclosure, the methods and/or algorithm(s) described herein can be performed/operated as software program(s) running on and/or by a computer processor. Furthermore, software implementations can include, but are not limited to, distributed processing and/or component/object distributed processing, parallel processing, and/or virtual machine processing.

FIG. 6 shows a block diagram of a cyberinfrastructure 500 according to some embodiments. As shown in FIG. 6, the cyberinfrastructure 500 can include an intelligent control 502, a processing unit 504, a memory unit/memory 506, a non-transitory computer-readable medium 508, a human machine interface (HMI) 510 wherein the HMI 510 can include a display 512, and a communications module 514. Each component of the cyberinfrastructure 500 can be operationally connected. For example, according to some embodiments, each component of the cyberinfrastructure 500 can be operatively connected, via a common bus and/or any other suitable connection element, such that all components of the cyberinfrastructure 500 can be in communication with each other.

The cyberinfrastructure 500 can include an intelligent control 502 (i.e., a controller) and components for establishing communications. Examples of such a controller may be processing units alone or other subcomponents of computing devices. The intelligent control 502 can also include other components and can be implemented partially or entirely on a semiconductor (e.g., a field-programmable gate array (“FPGA”)) chip, such as a chip developed through a register transfer level (“RTL”) design process.

The cyberinfrastructure 500 can include a processing unit 504, which can comprise a processor or multiple processors. A processor is an electronic circuit which performs operations on some external data source, usually memory or some other data stream. Nonlimiting examples of processors include a microprocessor, a microcontroller, an arithmetic logic unit (“ALU”), and most notably, a central processing unit (“CPU”). A CPU, also called a central processor or main processor, is the electronic circuitry within a computer that carries out the instructions of a computer program by performing the basic arithmetic, logic, controlling, and input/output (“I/O”) operations specified by the instructions. Processing units/processors are common in tablets, telephones, handheld devices, laptops, user displays, smart devices (TV, speaker, watch, etc.), and other computing devices. While the intelligent control 502 and the processing unit 504 are shown as separate components in FIG. 6, according to some embodiments the processing unit 504 can be incorporated into the intelligent control 502 or vice versa.

The cyberinfrastructure 500 can include a memory unit/memory 506. The memory 506 can include, in some embodiments, a program storage area and/or data storage area. The memory can comprise read-only memory (“ROM”, an example of non-volatile memory, meaning it does not lose data when it is not connected to a power source) or random access memory (“RAM”, an example of volatile memory, meaning it will lose its data when not connected to a power source). Nonlimiting examples of volatile memory include static RAM (“SRAM”), dynamic RAM (“DRAM”), synchronous DRAM (“SDRAM”), etc. Examples of non-volatile memory include electrically erasable programmable read only memory (“EEPROM”), flash memory, hard disks, SD cards, etc. In some embodiments, the processing unit 504, which can comprise a processor, a microprocessor, or a microcontroller, and/or the intelligent control 502 is operationally connected to the memory 506 and is configured to execute software instructions that are capable of being stored in the memory 506 and/or any other suitable location such as the non-transitory computer readable medium 508.

The cyberinfrastructure 500 can include a non-transitory computer-readable medium 508 (also known as a non-transitory computer-readable storage medium), which could be and/or comprise the memory unit/memory 506 described above. According to some embodiments, the non-transitory computer-readable medium 508 can store instructions that, when executed, may cause the system 100 and/or the system 400 to perform any method and/or algorithm described herein as well as control and/or operate any aspects of the system 100 and/or the system 400. While the memory 506 and non-transitory computer readable medium 508 are shown to be separate components in FIG. 6, the memory 506 and non-transitory computer readable medium 508 could be a single component and/or one could be incorporated into the other according to some embodiments. According to some embodiments, the intelligent control 502 and/or the processing unit 504 could be operationally connected to the non-transitory computer readable medium 508 such that the intelligent control 502 and/or the processing unit 504 can execute instructions that are stored on the non-transitory computer readable medium 508.

In communications and computing, a computer readable medium is a medium capable of storing data in a format readable by a mechanical device.

The non-transitory computer readable medium 508 can operate under control of an operating system stored in the memory unit/memory 506 or stored in any other location. The non-transitory computer readable medium 508 can implement a compiler which allows a software application written in a programming language such as COBOL, C++, FORTRAN, or any other known programming language to be translated into code readable by a processing unit such as the processing unit 504. After compilation, the intelligent control 502 and/or processing unit 504 can access and manipulate data stored in the memory of the non-transitory computer readable medium 508 using the relationships and logic dictated by the software application and generated using the compiler.

According to some embodiments, the software application and the compiler are tangibly embodied in the non-transitory computer-readable medium 508. When the instructions of the non-transitory computer readable medium 508 are read and executed, the cyberinfrastructure 500 can perform the steps necessary to implement and/or use any aspects of the present disclosure. According to some embodiments, the software application, operating instructions, and/or firmware (semi-permanent software programmed into read-only memory) may be tangibly embodied in the memory unit/memory 506, the non-transitory computer-readable medium 508, and/or data communication devices, thereby making the software application a product or article of manufacture according to the present disclosure.

The cyberinfrastructure 500 can further include a human machine interface (HMI) 510. The HMI 510, also known as a user interface, is how a user interacts with a machine. The HMI 510 can be a digital interface, a command-line interface, a graphical user interface (“GUI”), oral interface, virtual reality interface, or any other way a user can interact with a machine (user-machine interface). For example, the HMI 510 can include a combination of digital and/or analog input and/or output means and/or devices or any other type of user interface input/output device required to achieve a desired level of control and monitoring for a device and/or system, such as the system 100 and/or the system 400. Nonlimiting examples of input and/or output means include computer mice, keyboards, touchscreens, knobs, dials, switches, buttons, speakers, microphones, printers, LIDAR, RADAR, etc. Input(s) received by the HMI 510 can then be sent to the intelligent control 502, a microcontroller, and/or any type of controller to control operational aspects of a device and/or system such as the system 100 and/or the system 400.

The HMI 510 can include a display 512, which can act as an input and/or output device. More particularly, the display 512 can be a liquid crystal display (“LCD”), a light-emitting diode (“LED”) display, an organic LED (“OLED”) display, an electroluminescent display (“ELD”), a surface-conduction electron emitter display (“SED”), a field-emission display (“FED”), a thin-film transistor (“TFT”) LCD, a bistable cholesteric reflective display (i.e., e-paper), a touch-screen display, etc. The HMI 510 and/or display 512 also can be configured with the intelligent control 502 and/or another microcontroller to display conditions or data associated with the system 100, the system 400, and/or another device in real-time or substantially real-time.

According to some embodiments, a user can interact with the system 100 and/or the system 400 via the HMI 510 and/or the display 512. For example, according to some embodiments, a user can input, enter, and/or specify a target quality for the purified ethanol to achieve via the HMI 510 and/or the display 512.

The cyberinfrastructure 500 can include a communications module 514. The communications module 514 can include any combination of modem(s), router(s), access point(s), bridge(s), gateway(s), hub(s), repeater(s), switch(es), transceiver(s), and the like in order to facilitate communication. The communications module 514 can be configured to perform data communication wirelessly and/or in a wired fashion. The communications module 514 can include one or more communications ports such as Ethernet, serial advanced technology attachment (“SATA”), universal serial bus (“USB”), or integrated drive electronics (“IDE”), for transferring, sending, receiving, and/or or storing data.

According to some embodiments, the communications module 514 and/or other components of the system 100 and/or system 400 are able to perform data communication within the system 100, within the system 400, and/or externally of the system 100 and/or the system 400 in a wireless fashion using any sort of wireless connection device and/or protocol. This can include, but is not limited to, Bluetooth, Wi-Fi, cellular data, radio waves, satellite, and/or generally any other form of wireless connection. Therefore, the communications module 514 and/or any other component(s) of the system 100 and/or system 400 can include generally any electronic components necessary to allow for such wireless communication.

According to some embodiments, the communications module 514 and/or other components of the system 100 and/or the system 400 are able to perform data communication within the system 100, within the system 400, and/or externally of the system 100 and/or system 400 via a wired connection. Wired communication can take the form of CAN bus, Ethernet, co-axial cable, fiber optic line, and/or generally any other device and/or protocol which will allow for wired communication. Therefore, the communications module 514 and/or any other component(s) of the system 100 and/or the system 400 can include generally any electronic components necessary to allow for such wired communication.

While the cyberinfrastructure 500 can be included to perform, operate, and/or assist in the control and/or operation of aspects of the system 100, the system 400, method(s) described herein, and/or the algorithm 200, according to some embodiments, online analytical equipment can be included as part of the system 100 and/or as part of the system 400 in order to perform, operate, and/or assist in the control and/or operation of aspects of the system 100, the system 400, method(s) described herein, and/or the algorithm 200. The communications module 514, the intelligent control 502, and/or any other aspects of the system 100 and/or the system 400 can be used to communicate with the online analytical equipment.

The system 100 and/or system 400 can include whatever additional sensors, analytical equipment, and/or any other component(s) necessary to perform any aspects of the methods and/or algorithms described herein.

As described herein, online analytical equipment, can include, but is not limited to, gas chromatography and liquid chromatography (including, but not limited to, HPLC and UPLC).

It should be appreciated that each pump described herein can be configured to pump, move, and/or circulate fluid throughout the system 100 and/or the system 400. Additionally, each pump can be and/or comprise a peristaltic pump, a positive-displacement pump, a gear pump, a screw pump, a progressing cavity pump, a roots-type pump, a plunger pump, a rope pump, an impulse pump, a hydraulic pump, a velocity pump, a turbine pump, a gravity pump, a steam pump, a valveless pump, and/or any other suitable type of pump.

It should be appreciated that for any component of the system 100 described herein and/or shown in any of FIG. 1 that includes a fluid stream entering and/or exiting the component, said component can include a fluid entrance and/or exit. Such a fluid entrance and/or exit could be and/or comprise a nozzle, inlet, outlet, feed, drain, pipe, and/or any other kind of suitable opening. It should be appreciated that for any component of the system 400 described herein and/or shown in FIG. 2 that includes a fluid stream entering and/or exiting the component, said component can include a fluid entrance and/or exit. Such a fluid entrance and/or exit could be and/or comprise a nozzle, inlet, outlet, feed, drain, pipe, and/or any other kind of suitable opening.

It should be appreciated that the system 100 and the system 400 are configured to, and comprise the necessary components to, identify the types, categories, species, and quantities of impurities in the ethanol at any point/phase of the performance of the system 100 and/or the system 400. It should also be appreciated that the system 100 and the system 400 are configured to, and comprise the necessary components to, determine, measure, and/or identify boundaries in terms of percentages/concentrations of said impurities in the ethanol at any point/phase of the performance of the system 100 and/or the system 400.

The system 100, the algorithm 200, the system 400, the cyberinfrastructure 500, and/or any other method(s), algorithm(s), and/or aspects of performance/operation of the system 100 and/or the system 400 can be controlled and/or operated by simulation software. Additionally, any method, algorithm, software, and/or cyberinfrastructure described herein can incorporate and/or be incorporated into said simulation software. The simulation software can utilize sequential-modular or equation-based simulations. This simulation software can include the use of associated component and physical properties databases. Thus, according to some embodiments, the cyberinfrastructure 500 can include one or more databases comprising a structured set of data and/or information relating to associated component and physical properties. The one or more databases, as well as data and/or information contained therein, need not reside in a single physical or electronic location. For example, the one or more databases may reside, at least in part, on a local storage device, in an external hard drive, on a database server connected to a network, on a cloud-based storage system, in a distributed ledger (such as those commonly used with blockchain technology), or the like. The simulation software can optimize the performance/operation of the system 100, the algorithm 200, the system 400, the cyberinfrastructure 500, and/or any other method(s), algorithm(s), and/or aspects related to the performance/operation of the system 100 and/or the system 400 such that very low energy levels are consumed and such that the system 100 and/or the system 400 (and corresponding aspects such as the algorithm 200, cyberinfrastructure 500, etc.) are entirely driven with electricity. This allows for the system 100 (and all corresponding aspects) and/or the system 400 (and all corresponding aspects) to be controlled and/or operated via green power. Thus, this adds to the environmental friendliness of the system 100 and/or the system 400.

Therefore, as understood from the present disclosure, the system 100 and the system 400 as well as other aspects of the present disclosure including, but not limited to, the algorithm 200, cyberinfrastructure 500, and any other apparatus(es), method(s), algorithm(s), and/or system(s) described herein provide the ability to purify ethanol, such as fuel ethanol, such that the purified ethanol is suitable for use in the pharmaceutical, beverage, and laboratory industries. The system 100, the system 400, and other aspects of the present disclosure provide the ability to purify chemicals, including ethanol, in a cost-effective, energy-efficient, and environmentally friendly manner. The system 100, the system 400, and other aspects of the present disclosure provide the ability to purify chemicals, such as ethanol, without the use of filtration, extraction addition, or catalysts. The system 100, the system 400, and other aspects of the present disclosure provide the ability to purify chemicals, such as ethanol, such that the purified chemical is essentially absolutely pure, such that the impurity levels of the purified chemical are less than 10 ppm, and/or such that the amount of impurities is less than 10 grams per liter of ethanol. The system 100, the system 400, and other aspects of the present disclosure provide the ability to purify fuel ethanol such that the purified ethanol achieves a quality suitable for use as beverage alcohol.

### EXAMPLES

The following examples are offered by way of illustration and are not limitations on the inventions disclosed herein. It should be appreciated that ethanol plants vary in scale, system configuration, and other aspects. Accordingly, the inventions disclosed herein can apply to such variations and are not limited by the features disclosed in the following examples.

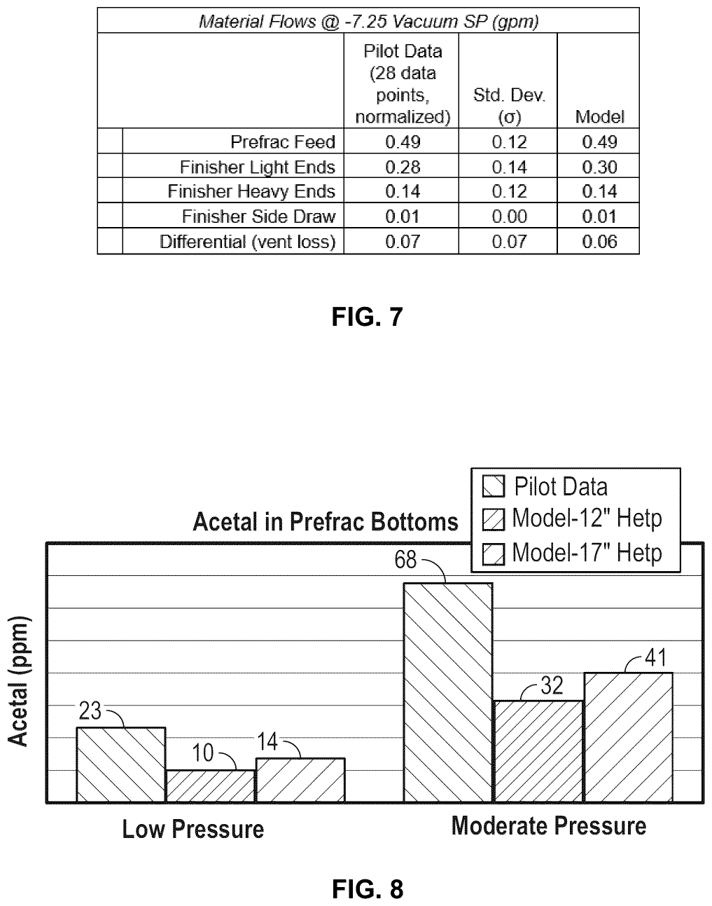

These examples illustrate results using a small scale pilot distillation unit representing the system 100. FIG. 7 shows a table illustrating the material flows throughout the pilot distillation unit. The material flows refer to the distribution of the feed flow to their ultimate destinations of light ends purge, heavy ends purge, final purified product, and vent loss. All feed and product flows were measured using a timed tote level decrease or increase method, which is an accurate way to provide the average inlet and outlet flows over a relatively short period of time such as one hour. The loss of product to the vacuum vent (an unmeasured flow) is determined by difference. The measured data provided in the table of FIG. 7 applies the median value instead of the mean value to reduce the influence of data that lies well outside of the normal distribution of data. For this reason, the raw measured flow data does not add up quite to 100%, although very close (97%). The (normalized) data provides reasonable material balance closure and is used in the model.

As shown in FIG. 7, for the pilot distillation unit, the measured normalized value for the prefractionation feed flow was 0.49, the measured normalized value for the finisher light ends flow was 0.28, the measured normalized value for the finisher heavy ends flow was 0.14, the measured normalized value for the finisher side draw flow was 0.01, and the measured normalized value for the differential (vent loss) flow was 0.07. Further, as shown in FIG. 7, the standard deviation value for prefractionation feed flow was 0.12, the standard deviation value for finisher light ends flow was 0.14, the standard deviation value for finisher heavy ends flow was 0.12, the standard deviation value for finisher side draw flow was 0.00, and the standard deviation value for differential (vent loss) flow was 0.07. Additionally, as shown in FIG. 7, for the model, the predicted normalized value for prefractionation feed flow was 0.49, the predicted normalized value for finisher light ends flow was 0.30, the predicted normalized value for finisher heavy ends flow was 0.14, the predicted normalized value for finisher side draw flow was 0.01, and the predicted normalized value for differential (vent loss) flow was 0.06. According to some embodiments, for the measured data, the measured normalized value for the prefractionation feed flow was 0.48, the measured normalized value for the finisher light ends flow was 0.27, the measured normalized value for the finisher heavy ends flow was 0.13, the measured normalized value for the finisher side draw flow was 0.01, and the measured normalized value for the differential (vent loss) flow was 0.07.

Using the results of the measured data analysis, the model set the feed, heavy ends, and side draw flows. The model also set the reboiler duty based on condensate flow measurements provided by the pilot distillation unit data. Subsequently, the model determined the light ends flow and the vent flow also via difference. As a mass-based crosscheck of the vent flow ethanol loss from the model, the estimated ethanol loss was 8% based on a 10 lbs/hr air leak rate (via the inert bleed) and an 80 degree Fahrenheit finisher reflux temperature.

As part of the data collection program, the pilot distillation unit also provided gas chromatograph (GC) analytical data for key streams of the pilot distillation unit, wherein such streams include: feed, prefractionation bottoms, reflux, finisher light ends, finisher heavy ends, and finisher side draw (high purity product). To simplify this analysis, only the light key component (acetal) and the heavy key component (n-propanol) were included in the comparison of measured results vs. model results. To note, all analyses were available for review and generally showed non-detectable concentrations in the side draw product for a full range of light and heavy ends impurities, which was also predicted by the model.

The experiments included measuring and/or considering measured results obtained from the pilot distillation unit described above for both a low and moderate pressure, predicted model results for a worst case model at both low and moderate pressure, and predicted model results for a best case model at both low and moderate pressure. Thus, experimentation included six examples. To define the case comparison matrix, “best case” assumed that the structured packing performs as expected by Sulzer's best estimate. In other words, a “best case” theoretical separation stage in each distillation column is equivalent to 12 inches of structured packing installed (12 inch height equivalent of a theoretical plate (HETP)). The “worst case” was arbitrarily defined by increasing the height equivalent theoretical stage to about 17 inches to test whether the packing performance agrees more closely to the measured data (17 inch height equivalent of a theoretical plate (HETP)). The term “moderate pressure” refers to data generated at 7 psia (a total of 7 data points) and “low pressure” refers to data generated at 3.75 psia.

### Example 1

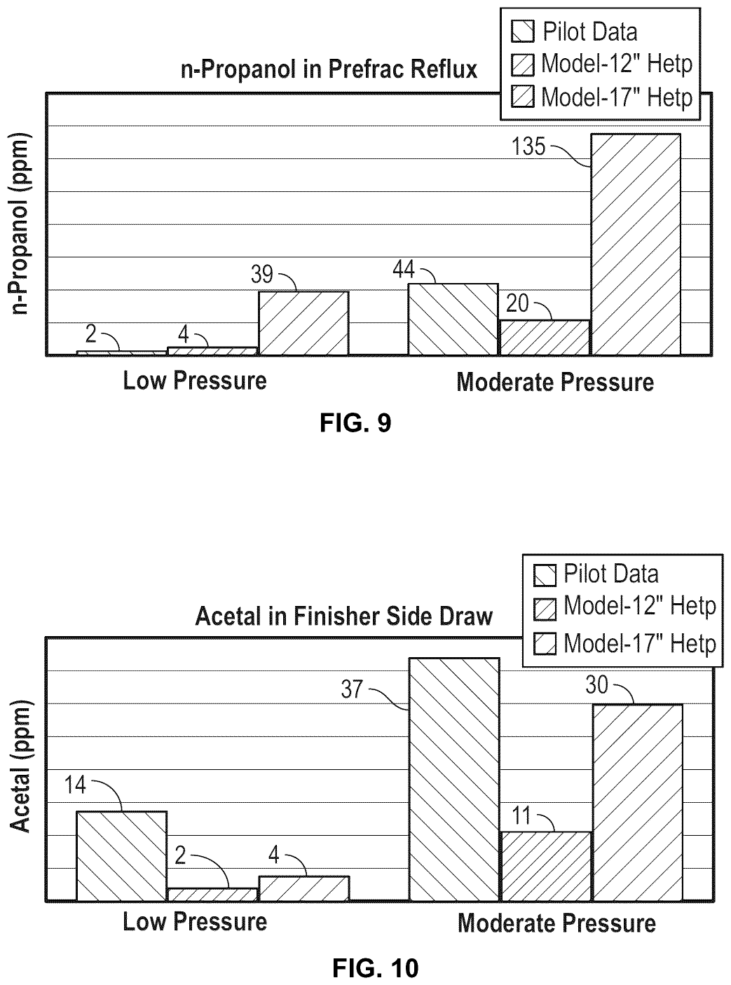

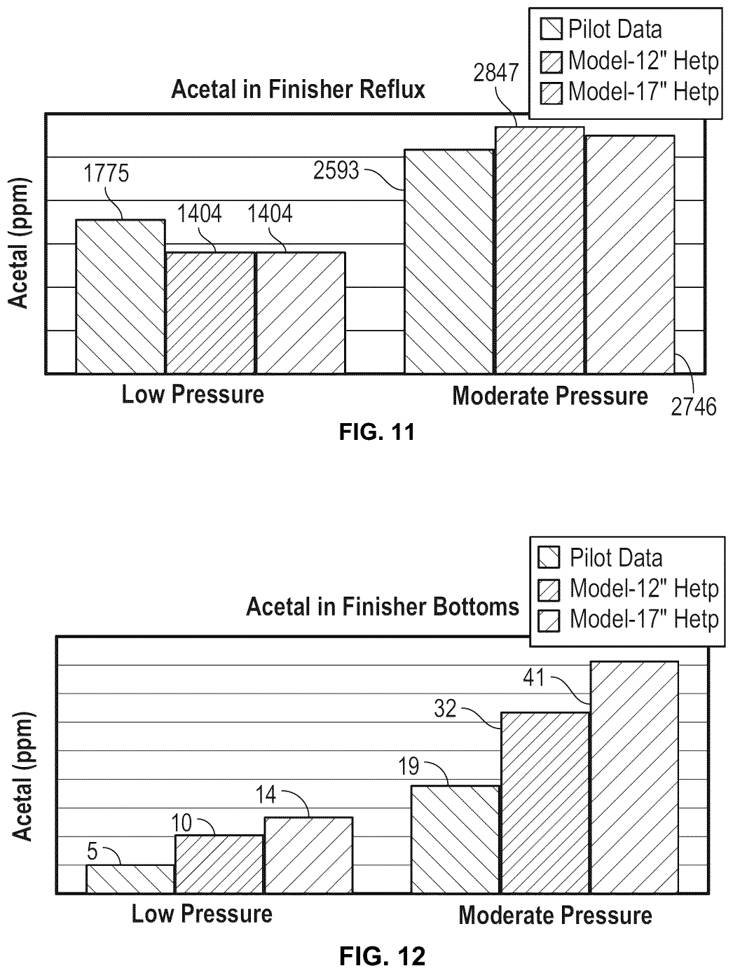

Measured data was collected for the pilot distillation unit at low pressure. The results are shown in FIGS. 8-12. At low pressure the amount of acetal in prefractionation bottoms for the pilot distillation unit was 23 ppm as shown in FIG. 8. At low pressure the amount of n-Propanol in prefractionation reflux for the pilot distillation unit was 2 ppm as shown in FIG. 9. At low pressure the amount of acetal in finisher side draw for the pilot distillation unit was 14 ppm as shown in FIG. 10. At low pressure the amount of acetal in finisher reflux for the pilot distillation unit was 1775 ppm as shown in FIG. 11. At low pressure the amount of acetal in finisher bottoms for the pilot distillation unit was 5 ppm as shown in FIG. 12.

### Example 2

Measured data was collected for the pilot distillation unit at moderate pressure. The results are shown in FIGS. 8-12. At moderate pressure the amount of acetal in prefractionation bottoms for the pilot distillation unit was 68 ppm as shown in FIG. 8. At moderate pressure the amount of n-Propanol in prefractionation reflux for the pilot distillation unit was 44 ppm as shown in FIG. 9. At moderate pressure the amount of acetal in finisher side draw for the pilot distillation unit was 37 ppm as shown in FIG. 10. At moderate pressure the amount of acetal in finisher reflux for the pilot distillation unit was 2593 ppm as shown in FIG. 11. At moderate pressure the amount of acetal in finisher bottoms for the pilot distillation unit was 19 ppm as shown in FIG. 12.

### Example 3

Model data was predicted for the best case model at low pressure. The results are shown in FIGS. 8-12. At low pressure the amount of acetal in prefractionation bottoms for the best case model was 10 ppm as shown in FIG. 8. At low pressure the amount of n-Propanol in prefractionation reflux for the best case model was 4 ppm as shown in FIG. 9. At low pressure the amount of acetal in finisher side draw for the best case model was 2 ppm as shown in FIG. 10. At low pressure the amount of acetal in finisher reflux for the best case model was 1404 ppm as shown in FIG. 11. At low pressure the amount of acetal in finisher bottoms for the best case model was 10 ppm as shown in FIG. 12.

### Example 4

Model data was predicted for the best case model at moderate pressure. The results are shown in FIGS. 8-12. At moderate pressure the amount of acetal in prefractionation bottoms for the best case model was 32 ppm as shown in FIG. 8. At moderate pressure the amount of n-Propanol in prefractionation reflux for the best case model was 20 ppm as shown in FIG. 9. At moderate pressure the amount of acetal in finisher side draw for the best case model was 11 ppm as shown in FIG. 10. At moderate pressure the amount of acetal in finisher reflux for the best case model was 2847 ppm as shown in FIG. 11. At moderate pressure the amount of acetal in finisher bottoms for the best case model was 32 ppm as shown in FIG. 12.

### Example 5

Model data was predicted for the worst case model at low pressure. The results are shown in FIGS. 8-12. At low pressure the amount of acetal in prefractionation bottoms for the worst case model was 14 ppm as shown in FIG. 8. At low pressure the amount of n-Propanol in prefractionation reflux for the worst case model was 39 ppm as shown in FIG. 9. At low pressure the amount of acetal in finisher side draw for the worst case model was 4 ppm as shown in FIG. 10. At low pressure the amount of acetal in finisher reflux for the worst case model was 1404 ppm as shown in FIG. 11. At low pressure the amount of acetal in finisher bottoms for the worst case model was 14 ppm as shown in FIG. 12.

### Example 6

Model data was predicted for the worst case model at moderate pressure. The results are shown in FIGS. 8-12. At moderate pressure the amount of acetal in prefractionation bottoms for the worst case model was 41 ppm as shown in FIG. 8. At moderate pressure the amount of n-Propanol in prefractionation reflux for the worst case model was 135 ppm as shown in FIG. 9. At moderate pressure the amount of acetal in finisher side draw for the worst case model was 30 ppm as shown in FIG. 10. At moderate pressure the amount of acetal in finisher reflux for the worst case model was 2746 ppm as shown in FIG. 11. At moderate pressure the amount of acetal in finisher bottoms for the worst case model was 41 ppm as shown in FIG. 12.

Based on Examples 1-6 and FIGS. 7-12, several conclusions become apparent. As shown in FIG. 8, operating at a lower pressure resulted in a 3× reduction in acetal in the prefractionation bottoms without significantly changing the operating conditions (tops/bottoms split, reflux or feed rate). The model, although predicting generally lower acetal concentrations, reflects this reduction almost exactly.

As shown in FIG. 9, the best case model result agreed reasonably well with the measured data of the pilot distillation unit, which indicates an improvement in n-propanol separation at the lower operating pressure. The worst case prediction broke significantly from the measured data of the pilot distillation unit for both pressure cases, suggesting the Sulzer packing performance estimate to be more accurate. There are no other graphical n-propanol comparisons provided as both the measurements and the model indicate zero n-propanol concentrations in the prefractionation reflux, the finisher reflux, and the finisher side draw at the conditions established by the pilot distillation unit.

As shown in FIG. 10, the model overpredicted the acetal concentration in finisher side draws in both the best and worst case model scenarios.

As shown in FIG. 11, the model provided reasonably good agreement of the concentrating effect of acetal in the finishing tower, providing confidence that the acetal volatility in the model is accurately represented.

As shown in FIG. 12, the measurements of acetal in finisher bottoms for the pilot distillation unit are lower than the model predictions. The best case model results compared to the measured data of the pilot distillation unit for both pressure cases are still reasonable.

The distillation concept (prefractionation followed by ethanol recovery in a finishing column) is generally confirmed to remove significant impurities from the ethanol, at least at low ethanol recovery rates.

The model generally agreed with measured data, although the model is a bit optimistic with respect to acetal separation.

The operating pressure of the distillation has a significant impact on light and heavy key separation, which is also confirmed by the model.

The Sum of Squares Deviation for the model data generated at 12 inch per theoretical stage was lower (better) than the 17 inch per theoretical stage for all comparisons. A single screening case assuming a 6 inch packing height per theoretical stage at 3.25 psia did not fit the data as well as the 12 inch assumption.

With a small adjustment to the equivalent height of a theoretical stage as a factor of safety, the model represents the purification process accurately.

From the foregoing, it can be seen that the present disclosure accomplishes at least all of the stated objectives.

What is claimed is:

1. A system for chemical purification, the system comprising:
a first tower, wherein the first tower is configured to receive a crude chemical;
a second tower operationally connected to the first tower, wherein the second tower is configured to output a purified chemical;
wherein each of the first and second towers operate based on independently adjustable tower operations; and
wherein the system is controlled by an algorithm wherein the algorithm comprises:
determining a quality of the crude chemical;
determining a target quality for the purified chemical; and
adjusting the tower operations so that the purified chemical achieves the target quality.

2. The system of claim 1, wherein the first tower is a prefractionation tower configured to receive the crude chemical at or near a middle portion and further configured to split heavy and light impurities wherein fluid comprising the heavy impurities and fluid comprising the light impurities are output from opposite ends of the first tower.

3. The system of claim 2, wherein the system is configured to identify categories and/or species of the heavy and light impurities, and wherein the system is further configured to set and/or identify boundaries in terms of percentages and/or concentrations of the heavy and light impurities.

4. The system of claim 2, wherein the second tower is a finishing tower configured to receive the fluid comprising light impurities from the first tower at a first end and the fluid comprising the heavy impurities from the first tower at a second end.

5. The system of claim 3, wherein the second tower is configured to output additional fluid comprising light impurities from the first end and output additional fluid comprising heavy impurities from the second end.

6. The system of claim 1, wherein the second tower is configured to perform a heart cut to beget the purified chemical, and wherein the second tower is further configured to output the purified chemical from a center portion of the second tower.

7. The system of claim 1, wherein the adjustable tower operations comprise depth of vacuum, distillate-to-feed ratio, reflux-to-distillate ratio, water addition to the crude chemical fed into the first tower, and bottoms rate.

8. The system of claim 1, wherein the algorithm comprises a feedforward algorithm, a feedback algorithm, and/or incorporates feedforward and feedback characteristics.

9. The system of claim 1, wherein gas chromatography is utilized to measure and/or determine the quality of the crude chemical and an actual quality of the purified chemical.

10. The system of claim 1, wherein the system utilizes mechanical vapor recompression.

11. The system of claim 1, wherein each of the first and second towers are packed and/or trayed.

12. The system of claim 1, wherein the crude chemical is crude ethanol.

13. The system of claim 1, wherein the purified chemical is purified ethanol wherein the purified ethanol is nearly absolutely pure.

14. The system of claim 1, wherein the purified chemical comprises impurity levels that are less than 10 parts per million.

15. A method of refining a chemical, the method comprising:
determining a quality of a crude chemical;
determining a target quality for a refined chemical;
adjusting column operations of a first and second column so that the refined chemical achieves the target quality;
inputting the crude chemical into the first column;
performing prefractionation of the crude chemical;
separately outputting fractioned portions of the crude chemical from the first column;
separately inputting the fractioned portions of the crude chemical into the second column;
refining the fractioned portions of the crude chemical via the second column to beget a refined chemical; and
outputting the refined chemical from the second column.

16. The method of claim 15, wherein performing prefractionation of the crude chemical comprises splitting the crude chemical into fluid comprising heavy impurities and fluid comprising light impurities.

17. The method of claim 16, wherein refining the fractioned portions of the crude chemical comprises performing a heart cut of the fluid comprising the heavy impurities and the fluid comprising the light impurities.

18. The method of claim 15, further comprising customizing the target quality for the refined chemical.

19. The system of claim 15, further comprising verifying an actual quality of the refined chemical to ensure compliance with the target quality.

20. A chemical distillation assembly, the assembly comprising:
a first tower, wherein the first tower is configured to receive a crude chemical and perform prefractionation of said crude chemical;
a second tower operationally connected to the first tower, wherein the second tower is configured to receive fractioned crude chemical from the first tower, distill the fractioned crude chemical to obtain a distilled chemical, and to output the distilled chemical;
wherein each of the first and second towers operate based on independently adjustable tower operations; and
wherein the assembly is controlled by an algorithm wherein the algorithm comprises:
measuring a quality of the crude chemical;
acquiring a target quality for the distilled chemical wherein the target quality for the distilled chemical is based on user input; and
automatically adjusting the tower operations so that the distilled chemical achieves the target quality.
